SEQUENCE LISTING (70 sequences)

SEQ ID NO: 1 - DNA, 723 bp, synthetic construct
  misc_feature 1..723: note = NGFwt optimized coding region sequence
  source 1..723: mol_type = other DNA
atgtccatgttgttctacactctgatcacagcttttctgatcggcatacaggcggaaccacactcagagagcaatgtccctgcaggacacaccatcccccaagcccactggactaaacttcagcattcccttgacactgcccttcgcagagcccgcagcgccccggcagcggcgatagctgcacgcgtggcggggcagacccgcaacattactgtggaccccaggctgtttaaaaagcggcgactccgttcaccccgtgtgctgtttagcacccagcctccccgtgaagctgcagacactcaggatctggacttcgaggtcggtggtgctgcccccttcaacaggactcacaggagcaagcggtcatcatcccatcccatcttccacaggggcgaattctcggtgtgtgacagtgtcagcgtgtgggttggggataagaccaccgccacagacatcaagggcaaggaggtgatggtgttgggagaggtgaacattaacaacagtgtattcaaacagtacttttttgagaccaagtgccgggacccaaatcccgttgacagcgggtgccggggcattgactcaaagcactggaactcatattgtaccacgactcacacctttgtcaaggcgctgaccatggatggcaagcaggctgcctggcggtttatccggatagatacggcctgtgtgtgtgtgctcagcaggaaggctgtgagaagagcc

SEQ ID NO: 2 - DNA, 723 bp, synthetic construct
  misc_feature 1..723: note = NGFmut optimized coding region sequence
  source 1..723: mol_type = other DNA
atgtccatgttgttctacacactgatcaccgccttcctgatcggcatccaggccgagcctcacagcgagtccaatgtgcccgccggccacaccatcccccaggctcattggaccaagctgcagcactccctggatacagccctgagaagagccagatccgcccctgccgccgccatcgctgctagagtggccggacagacaaggaacatcacagtggaccctagactgtttaagaagagaagactgaggagccccagagtgctgttctccacacagccccccagagaggccgccgacacacaggacctggatttcgaggtgggcggcgccgccccttttaacagaacacacagatccaagaggtccagctcccaccctatcttccacagaggcgagttcagcgtgtgcgactccgtgagcgtgtgggtgggcgacaagaccaccgccaccgacatcaagggcaaggaggtcatggtgctgggcgaggtgaacatcaataacagcgtgtttaagcagtactttttcgagaccaagtgcagagaccctaaccctgtggactccggctgtagaggcatcgactccaagcactggaatagctactgcaccacaacccacacattcgtgaaggccctgacaatggacggcaagcaggccgcctggtggttcatcagaatcgacaccgcctgtgtgtgtgtgctgagcagaaaggctgtgagaagagcc

SEQ ID NO: 3 - DNA, 762 bp, synthetic construct
  misc_feature 1..762: note = IGK19-NGFmut optimized sequence of coding region
  source 1..762: mol_type = other DNA
atggagacagacacactcctgctatgggtactgctgctctgggttccaggttccaccggagacgagcctcacagcgagtccaatgtgcccgccggccacaccatcccccaggctcattggaccaagctgcagcactccctggatacagccctgagaagagccagatccgcccctgccgccgccatcgctgctagagtggccggacagacaaggaacatcacagtggaccctagactgtttaagaagagaagactgaggagccccagagtgctgttctccacacagccccccagagaggccgccgacacacaggacctggatttcgaggtgggcggcgccgccccttttaacagaacacacagatccaagaggtccagctcccaccctatcttccacagaggcgagttcagcgtgtgcgactccgtgagcgtgtgggtgggcgacaagaccaccgccaccgacatcaagggcaaggaggtcatggtgctgggcgaggtgaacatcaataacagcgtgtttaagcagtactttttcgagaccaagtgcagagaccctaaccctgtggactccggctgtagaggcatcgactccaagcactggaatagctactgcaccacaacccacacattcgtgaaggccctgacaatggacggcaagcaggccgcctggagattcatcagaatcgacaccgcctgtgtgtgtgtgctgagcagaaaggccgtgagaagggcctacccgtacgacgtgccggactacgcctga

SEQ ID NO: 4 - DNA, 423 bp, synthetic construct
  misc_feature 1..423: note = IGK122-NGFmut optimized sequence of coding region
  source 1..423: mol_type = other DNA
atggagacagacacactcctgctatgggtactgctgctctgggttccaggttccaccggagactccagctcccaccctatcttccacagaggcgagttcagcgtgtgcgactccgtgagcgtgtgggtgggcgacaagaccaccgccaccgacatcaagggcaaggaggtcatggtgctgggcgaggtgaacatcaataacagcgtgtttaagcagtactttttcgagaccaagtgcagagaccctaaccctgtggactccggctgtagaggcatcgactccaagcactggaatagctactgcaccacaacccacacattcgtgaaggccctgacaatggacggcaagcaggccgcctggtggttcatcagaatcgacaccgcctgtgtgtgtgtgctgagcagaaaggctgtgagaagagcc

SEQ ID NO: 5 - DNA, 939 bp, synthetic construct
  misc_feature 1..939: note = IGK19-NGF-PDGFRmut optimized sequence of coding region
  source 1..939: mol_type = other DNA
atggagacagacacactcctgctatgggtactgctgctctgggttccaggttccaccggagacgagcctcacagcgagtccaatgtgcccgccggccacaccatcccccaggctcattggaccaagctgcagcactccctggatacagccctgagaagagccagatccgcccctgccgccgccatcgctgctagagtggccggacagacaaggaacatcacagtggaccctagactgtttaagaagagaagactgaggagccccagagtgctgttctccacacagccccccagagaggccgccgacacacaggacctggatttcgaggtgggcggcgccgccccttttaacagaacacacagatccaagaggtccagctcccaccctatcttccacagaggcgagttcagcgtgtgcgactccgtgagcgtgtgggtgggcgacaagaccaccgccaccgacatcaagggcaaggaggtcatggtgctgggcgaggtgaacatcaataacagcgtgtttaagcagtactttttcgagaccaagtgcagagaccctaaccctgtggactccggctgtagaggcatcgactccaagcactggaatagctactgcaccacaacccacacattcgtgaaggccctgacaatggacggcaagcaggccgcctggagattcatcagaatcgacaccgcctgtgtgtgtgtgctgagcagaaaggccgtgagaagggcctacccgtacgacgtgccggactacgcctgatacccgtacgacgtgccggactacgccaatgctgtgggccaggacacgcaggaggtcatcgtggtgccacactccttgccctttaaggtggtggtgatctcagccatcctggccctggtggtgctcaccatcatctcccttatcatcctcatcatgctttggcagaagaagccacgt

SEQ ID NO: 6 - DNA, 600 bp, synthetic construct
  misc_feature 1..600: note = IGK122-NGF-PDGFRmut optimized sequence of coding region
  source 1..600: mol_type = other DNA
atggagacagacacactcctgctatgggtactgctgctctgggttccaggttccaccggagactccagctcccaccctatcttccacagaggcgagttcagcgtgtgcgactccgtgagcgtgtgggtgggcgacaagaccaccgccaccgacatcaagggcaaggaggtcatggtgctgggcgaggtgaacatcaataacagcgtgtttaagcagtactttttcgagaccaagtgcagagaccctaaccctgtggactccggctgtagaggcatcgactccaagcactggaatagctactgcaccacaacccacacattcgtgaaggccctgacaatggacggcaagcaggccgcctggtggttcatcagaatcgacaccgcctgtgtgtgtgtgctgagcagaaaggctgtgagaagagcctacccgtacgacgtgccggactacgccaatgctgtgggccaggacacgcaggaggtcatcgtggtgccacactccttgccctttaaggtggtggtgatctcagccatcctggccctggtggtgctcaccatcatctcccttatcatcctcatcatgctttggcagaagaagccacgt

SEQ ID NO: 7 - DNA, 723 bp, synthetic construct
  misc_feature 1..723: note = BDNF19-NGFmut optimized coding region sequence
  source 1..723: mol_type = other DNA
atgaccatcctgttcctgacaatggtcattagctacttcggctgcatgaaggccgagcctcacagcgagtccaatgtgcccgccggccacaccatcccccaggctcattggaccaagctgcagcactccctggatacagccctgagaagagccagatccgcccctgccgccgccatcgctgctagagtggccggacagacaaggaacatcacagtggaccctagactgtttaagaagagaagactgaggagccccagagtgctgttctccacacagccccccagagaggccgccgacacacaggacctggatttcgaggtgggcggcgccgccccttttaacagaacacacagatccaagaggtccagctcccaccctatcttccacagaggcgagttcagcgtgtgcgactccgtgagcgtgtgggtgggcgacaagaccaccgccaccgacatcaagggcaaggaggtcatggtgctgggcgaggtgaacatcaataacagcgtgtttaagcagtactttttcgagaccaagtgcagagaccctaaccctgtggactccggctgtagaggcatcgactccaagcactggaatagctactgcaccacaacccacacattcgtgaaggccctgacaatggacggcaagcaggccgcctggtggttcatcagaatcgacaccgcctgtgtgtgtgtgctgagcagaaaggctgtgagaagagcc

SEQ ID NO: 8 - DNA, 744 bp, synthetic construct
  misc_feature 1..744: note = BDNF122-NGFmut optimized coding region sequence
  source 1..744: mol_type = other DNA
atgaccatcctgttcctgacaatggtcattagctacttcggctgcatgaaggccgcccccatgaaggaggccaacatcagaggccagggcggcctggcctaccccggagtgagaacacacggcacactggagtccgtgaacggccccaaggccggctccaggggactgacaagcctggccgataccttcgagcacgtgatcgaggagctgctggacgaggaccagaaggtgaggcccaatgaggagaataataaggacgccgacctgtacacctccagggtcatgctgagcagccaggtgcccctggagcccccactgctgttcctgctggaggagtacaagaattacctggacgccgccaatatgtccatgagagtgagaaggtccagctcccaccctatcttccacagaggcgagttcagcgtgtgcgactccgtgagcgtgtgggtgggcgacaagaccaccgccaccgacatcaagggcaaggaggtcatggtgctgggcgaggtgaacatcaataacagcgtgtttaagcagtactttttcgagaccaagtgcagagaccctaaccctgtggactccggctgtagaggcatcgactccaagcactggaatagctactgcaccacaacccacacattcgtgaaggccctgacaatggacggcaagcaggccgcctggtggttcatcagaatcgacaccgcctgtgtgtgtgtgctgagcagaaaggctgtgagaagagcc

SEQ ID NO: 9 - DNA, 726 bp, synthetic construct
  misc_feature 1..726: note = NGFwt coding region sequence
  source 1..726: mol_type = other DNA
atgtccatgttgttctacactctgatcacagcttttctgatcggcatacaggcggaaccacactcagagagcaatgtccctgcaggacacaccatcccccaagcccactggactaaacttcagcattcccttgacactgcccttcgcagagcccgcagcgccccggcagcggcgatagctgcacgcgtggcggggcagacccgcaacattactgtggaccccaggctgtttaaaaagcggcgactccgttcaccccgtgtgctgtttagcacccagcctccccgtgaagctgcagacactcaggatctggacttcgaggtcggtggtgctgcccccttcaacaggactcacaggagcaagcggtcatcatcccatcccatcttccacaggggcgaattctcggtgtgtgacagtgtcagcgtgtgggttggggataagaccaccgccacagacatcaagggcaaggaggtgatggtgttgggagaggtgaacattaacaacagtgtattcaaacagtacttttttgagaccaagtgccgggacccaaatcccgttgacagcgggtgccggggcattgactcaaagcactggaactcatattgtaccacgactcacacctttgtcaaggcgctgaccatggatggcaagcaggctgcctggcggtttatccggatagatacggcctgtgtgtgtgtgctcagcaggaaggctgtgagaagagcctga

SEQ ID NO: 10 - DNA, 726 bp, synthetic construct
  misc_feature 1..726: note = NGFmut coding region sequence
  source 1..726: mol_type = other DNA
atgtccatgttgttctacactctgatcacagcttttctgatcggcatacaggcggaaccacactcagagagcaatgtccctgcaggacacaccatcccccaagcccactggactaaacttcagcattcccttgacactgcccttcgcagagcccgcagcgccccggcagcggcgatagctgcacgcgtggcggggcagacccgcaacattactgtggaccccaggctgtttaaaaagcggcgactccgttcaccccgtgtgctgtttagcacccagcctccccgtgaagctgcagacactcaggatctggacttcgaggtcggtggtgctgcccccttcaacaggactcacaggagcaagcggtcatcatcccatcccatcttccacaggggcgaattctcggtgtgtgacagtgtcagcgtgtgggttggggataagaccaccgccacagacatcaagggcaaggaggtgatggtgttgggagaggtgaacattaacaacagtgtattcaaacagtacttttttgagaccaagtgccgggacccaaatcccgttgacagcgggtgccggggcattgactcaaagcactggaactcatattgtaccacgactcacacctttgtcaaggcgctgaccatggatggcaagcaggctgcctggtggtttatccggatagatacggcctgtgtgtgtgtgctcagcaggaaggctgtgagaagagcctga

SEQ ID NO: 11 - DNA, 735 bp, synthetic construct
  misc_feature 1..735: note = IGK19-NGFmut coding region sequence
  source 1..735: mol_type = other DNA
atggagacagacacactcctgctatgggtactgctgctctgggttccaggttccactggtgacgaaccacactcagagagcaatgtccctgcaggacacaccatcccccaagcccactggactaaacttcagcattcccttgacactgcccttcgcagagcccgcagcgccccggcagcggcgatagctgcacgcgtggcggggcagacccgcaacattactgtggaccccaggctgtttaaaaagcggcgactccgttcaccccgtgtgctgtttagcacccagcctccccgtgaagctgcagacactcaggatctggacttcgaggtcggtggtgctgcccccttcaacaggactcacaggagcaagcggtcatcatcccatcccatcttccacaggggcgaattctcggtgtgtgacagtgtcagcgtgtgggttggggataagaccaccgccacagacatcaagggcaaggaggtgatggtgttgggagaggtgaacattaacaacagtgtattcaaacagtacttttttgagaccaagtgccgggacccaaatcccgttgacagcgggtgccggggcattgactcaaagcactggaactcatattgtaccacgactcacacctttgtcaaggcgctgaccatggatggcaagcaggctgcctggtggtttatccggatagatacggcctgtgtgtgtgtgctcagcaggaaggctgtgagaagagcctga

SEQ ID NO: 12 - DNA, 726 bp, synthetic construct
  misc_feature 1..726: note = BDNF19-NGFmut coding region sequence
  source 1..726: mol_type = other DNA
atgaccatccttttccttactatggttatttcatactttggttgcatgaaggctgaaccacactcagagagcaatgtccctgcaggacacaccatcccccaagcccactggactaaacttcagcattcccttgacactgcccttcgcagagcccgcagcgccccggcagcggcgatagctgcacgcgtggcggggcagacccgcaacattactgtggaccccaggctgtttaaaaagcggcgactccgttcaccccgtgtgctgtttagcacccagcctccccgtgaagctgcagacactcaggatctggacttcgaggtcggtggtgctgcccccttcaacaggactcacaggagcaagcggtcatcatcccatcccatcttccacaggggcgaattctcggtgtgtgacagtgtcagcgtgtgggttggggataagaccaccgccacagacatcaagggcaaggaggtgatggtgttgggagaggtgaacattaacaacagtgtattcaaacagtacttttttgagaccaagtgccgggacccaaatcccgttgacagcgggtgccggggcattgactcaaagcactggaactcatattgtaccacgactcacacctttgtcaaggcgctgaccatggatggcaagcaggctgcctggtggtttatccggatagatacggcctgtgtgtgtgtgctcagcaggaaggctgtgagaagagcctga

SEQ ID NO: 13 - protein, 241 aa, synthetic construct
  REGION 1..241: note = NGFwt amino acid sequence
MSMLFYTLITAFLIGIQAEPHSESNVPAGHTIPQAHWTKLQHSLDTALRRARSAPAAAIAARVAGQTRNITVDPRLFKKRRLRSPRVLFSTQPPREAADTQDLDFEVGGAAPFNRTHRSKRSSSHPIFHRGEFSVCDSVSVWVGDKTTATDIKGKEVMVLGEVNINNSVFKQYFFETKCRDPNPVDSGCRGIDSKHWNSYCTTTHTFVKALTMDGKQAAWRFIRIDTACVCVLSRKAVRRA

SEQ ID NO: 14 - protein, 241 aa, synthetic construct
  REGION 1..241: note = NGFmut amino acid sequence
MSMLFYTLITAFLIGIQAEPHSESNVPAGHTIPQAHWTKLQHSLDTALRRARSAPAAAIAARVAGQTRNITVDPRLFKKRRLRSPRVLFSTQPPREAADTQDLDFEVGGAAPFNRTHRSKRSSSHPIFHRGEFSVCDSVSVWVGDKTTATDIKGKEVMVLGEVNINNSVFKQYFFETKCRDPNPVDSGCRGIDSKHWNSYCTTTHTFVKALTMDGKQAAWWFIRIDTACVCVLSRKAVRRA

SEQ ID NO: 15 - protein, 253 aa, synthetic construct
  REGION 1..253: note = IGK19-NGFmut amino acid sequence
METDTLLLWVLLLWVPGSTGDEPHSESNVPAGHTIPQAHWTKLQHSLDTALRRARSAPAAAIAARVAGQTRNITVDPRLFKKRRLRSPRVLFSTQPPREAADTQDLDFEVGGAAPFNRTHRSKRSSSHPIFHRGEFSVCDSVSVWVGDKTTATDIKGKEVMVLGEVNINNSVFKQYFFETKCRDPNPVDSGCRGIDSKHWNSYCTTTHTFVKALTMDGKQAAWWFIRIDTACVCVLSRKAVRRAYPYDVPDYA

SEQ ID NO: 16 - protein, 141 aa, synthetic construct
  REGION 1..141: note = IGK122-NGFmut amino acid sequence
METDTLLLWVLLLWVPGSTGDSSSHPIFHRGEFSVCDSVSVWVGDKTTATDIKGKEVMVLGEVNINNSVFKQYFFETKCRDPNPVDSGCRGIDSKHWNSYCTTTHTFVKALTMDGKQAAWWFIRIDTACVCVLSRKAVRRA

SEQ ID NO: 17 - protein, 312 aa, synthetic construct
  REGION 1..312: note = IGK19-NGF-PDGFRmut amino acid sequence
METDTLLLWVLLLWVPGSTGDEPHSESNVPAGHTIPQAHWTKLQHSLDTALRRARSAPAAAIAARVAGQTRNITVDPRLFKKRRLRSPRVLFSTQPPREAADTQDLDFEVGGAAPFNRTHRSKRSSSHPIFHRGEFSVCDSVSVWVGDKTTATDIKGKEVMVLGEVNINNSVFKQYFFETKCRDPNPVDSGCRGIDSKHWNSYCTTTHTFVKALTMDGKQAAWWFIRIDTACVCVLSRKAVRRAYPYDVPDYAYPYDVPDYANAVGQDTQEVIVVPHSLPFKVVVISAILALVVLTIISLIILIMLWQKKPR

SEQ ID NO: 18 - protein, 200 aa, synthetic construct
  REGION 1..200: note = IGK122-NGF-PDGFRmut amino acid sequence
METDTLLLWVLLLWVPGSTGDSSSHPIFHRGEFSVCDSVSVWVGDKTTATDIKGKEVMVLGEVNINNSVFKQYFFETKCRDPNPVDSGCRGIDSKHWNSYCTTTHTFVKALTMDGKQAAWWFIRIDTACVCVLSRKAVRRAYPYDVPDYANAVGQDTQEVIVVPHSLPFKVVVISAILALVVLTIISLIILIMLWQKKPR

SEQ ID NO: 19 - protein, 241 aa, synthetic construct
  REGION 1..241: note = BDNF19-NGFmut amino acid sequence
MTILFLTMVISYFGCMKAEPHSESNVPAGHTIPQAHWTKLQHSLDTALRRARSAPAAAIAARVAGQTRNITVDPRLFKKRRLRSPRVLFSTQPPREAADTQDLDFEVGGAAPFNRTHRSKRSSSHPIFHRGEFSVCDSVSVWVGDKTTATDIKGKEVMVLGEVNINNSVFKQYFFETKCRDPNPVDSGCRGIDSKHWNSYCTTTHTFVKALTMDGKQAAWWFIRIDTACVCVLSRKAVRRA

SEQ ID NO: 20 - protein, 248 aa, synthetic construct
  REGION 1..248: note = BDNF122-NGFmut amino acid sequence
MTILFLTMVISYFGCMKAAPMKEANIRGQGGLAYPGVRTHGTLESVNGPKAGSRGLTSLADTFEHVIEELLDEDQKVRPNEENNKDADLYTSRVMLSSQVPLEPPLLFLLEEYKNYLDAANMSMRVRRSSSHPIFHRGEFSVCDSVSVWVGDKTTATDIKGKEVMVLGEVNINNSVFKQYFFETKCRDPNPVDSGCRGIDSKHWNSYCTTTHTFVKALTMDGKQAAWWFIRIDTACVCVLSRKAVRRA

SEQ ID NO: 21 - RNA, 723 nt, synthetic construct
  misc_feature 1..723: note = NGFwt mRNA sequence
  source 1..723: mol_type = other RNA
atgtccatgttgttctacactctgatcacagcttttctgatcggcatacaggcggaaccacactcagagagcaatgtccctgcaggacacaccatcccccaagcccactggactaaacttcagcattcccttgacactgcccttcgcagagcccgcagcgccccggcagcggcgatagctgcacgcgtggcggggcagacccgcaacattactgtggaccccaggctgtttaaaaagcggcgactccgttcaccccgtgtgctgtttagcacccagcctccccgtgaagctgcagacactcaggatctggacttcgaggtcggtggtgctgcccccttcaacaggactcacaggagcaagcggtcatcatcccatcccatcttccacaggggcgaattctcggtgtgtgacagtgtcagcgtgtgggttggggataagaccaccgccacagacatcaagggcaaggaggtgatggtgttgggagaggtgaacattaacaacagtgtattcaaacagtacttttttgagaccaagtgccgggacccaaatcccgttgacagcgggtgccggggcattgactcaaagcactggaactcatattgtaccacgactcacacctttgtcaaggcgctgaccatggatggcaagcaggctgcctggcggtttatccggatagatacggcctgtgtgtgtgtgctcagcaggaaggctgtgagaagagcc

SEQ ID NO: 22 - RNA, 723 nt, synthetic construct
  misc_feature 1..723: note = NGFmut mRNA sequence
  source 1..723: mol_type = other RNA
atgtccatgttgttctacacactgatcaccgccttcctgatcggcatccaggccgagcctcacagcgagtccaatgtgcccgccggccacaccatcccccaggctcattggaccaagctgcagcactccctggatacagccctgagaagagccagatccgcccctgccgccgccatcgctgctagagtggccggacagacaaggaacatcacagtggaccctagactgtttaagaagagaagactgaggagccccagagtgctgttctccacacagccccccagagaggccgccgacacacaggacctggatttcgaggtgggcggcgccgccccttttaacagaacacacagatccaagaggtccagctcccaccctatcttccacagaggcgagttcagcgtgtgcgactccgtgagcgtgtgggtgggcgacaagaccaccgccaccgacatcaagggcaaggaggtcatggtgctgggcgaggtgaacatcaataacagcgtgtttaagcagtactttttcgagaccaagtgcagagaccctaaccctgtggactccggctgtagaggcatcgactccaagcactggaatagctactgcaccacaacccacacattcgtgaaggccctgacaatggacggcaagcaggccgcctggtggttcatcagaatcgacaccgcctgtgtgtgtgtgctgagcagaaaggctgtgagaagagcc

SEQ ID NO: 23 - RNA, 762 nt, synthetic construct
  misc_feature 1..762: note = IGK19-NGFmut mRNA sequence
  source 1..762: mol_type = other RNA
atggagacagacacactcctgctatgggtactgctgctctgggttccaggttccaccggagacgagcctcacagcgagtccaatgtgcccgccggccacaccatcccccaggctcattggaccaagctgcagcactccctggatacagccctgagaagagccagatccgcccctgccgccgccatcgctgctagagtggccggacagacaaggaacatcacagtggaccctagactgtttaagaagagaagactgaggagccccagagtgctgttctccacacagccccccagagaggccgccgacacacaggacctggatttcgaggtgggcggcgccgccccttttaacagaacacacagatccaagaggtccagctcccaccctatcttccacagaggcgagttcagcgtgtgcgactccgtgagcgtgtgggtgggcgacaagaccaccgccaccgacatcaagggcaaggaggtcatggtgctgggcgaggtgaacatcaataacagcgtgtttaagcagtactttttcgagaccaagtgcagagaccctaaccctgtggactccggctgtagaggcatcgactccaagcactggaatagctactgcaccacaacccacacattcgtgaaggccctgacaatggacggcaagcaggccgcctggagattcatcagaatcgacaccgcctgtgtgtgtgtgctgagcagaaaggccgtgagaagggcctacccgtacgacgtgccggactacgcctga

SEQ ID NO: 24 - RNA, 423 nt, synthetic construct
  misc_feature 1..423: note = IGK122-NGFmut mRNA sequence
  source 1..423: mol_type = other RNA
atggagacagacacactcctgctatgggtactgctgctctgggttccaggttccaccggagactccagctcccaccctatcttccacagaggcgagttcagcgtgtgcgactccgtgagcgtgtgggtgggcgacaagaccaccgccaccgacatcaagggcaaggaggtcatggtgctgggcgaggtgaacatcaataacagcgtgtttaagcagtactttttcgagaccaagtgcagagaccctaaccctgtggactccggctgtagaggcatcgactccaagcactggaatagctactgcaccacaacccacacattcgtgaaggccctgacaatggacggcaagcaggccgcctggtggttcatcagaatcgacaccgcctgtgtgtgtgtgctgagcagaaaggctgtgagaagagcc

SEQ ID NO: 25 - RNA, 939 nt, synthetic construct
  misc_feature 1..939: note = IGK19-NGF-PDGFRmut mRNA sequence
  source 1..939: mol_type = other RNA
atggagacagacacactcctgctatgggtactgctgctctgggttccaggttccaccggagacgagcctcacagcgagtccaatgtgcccgccggccacaccatcccccaggctcattggaccaagctgcagcactccctggatacagccctgagaagagccagatccgcccctgccgccgccatcgctgctagagtggccggacagacaaggaacatcacagtggaccctagactgtttaagaagagaagactgaggagccccagagtgctgttctccacacagccccccagagaggccgccgacacacaggacctggatttcgaggtgggcggcgccgccccttttaacagaacacacagatccaagaggtccagctcccaccctatcttccacagaggcgagttcagcgtgtgcgactccgtgagcgtgtgggtgggcgacaagaccaccgccaccgacatcaagggcaaggaggtcatggtgctgggcgaggtgaacatcaataacagcgtgtttaagcagtactttttcgagaccaagtgcagagaccctaaccctgtggactccggctgtagaggcatcgactccaagcactggaatagctactgcaccacaacccacacattcgtgaaggccctgacaatggacggcaagcaggccgcctggagattcatcagaatcgacaccgcctgtgtgtgtgtgctgagcagaaaggccgtgagaagggcctacccgtacgacgtgccggactacgcctgatacccgtacgacgtgccggactacgccaatgctgtgggccaggacacgcaggaggtcatcgtggtgccacactccttgccctttaaggtggtggtgatctcagccatcctggccctggtggtgctcaccatcatctcccttatcatcctcatcatgctttggcagaagaagccacgt

SEQ ID NO: 26 - RNA, 600 nt, synthetic construct
  misc_feature 1..600: note = IGK122-NGF-PDGFRmut mRNA sequence
  source 1..600: mol_type = other RNA
atggagacagacacactcctgctatgggtactgctgctctgggttccaggttccaccggagactccagctcccaccctatcttccacagaggcgagttcagcgtgtgcgactccgtgagcgtgtgggtgggcgacaagaccaccgccaccgacatcaagggcaaggaggtcatggtgctgggcgaggtgaacatcaataacagcgtgtttaagcagtactttttcgagaccaagtgcagagaccctaaccctgtggactccggctgtagaggcatcgactccaagcactggaatagctactgcaccacaacccacacattcgtgaaggccctgacaatggacggcaagcaggccgcctggtggttcatcagaatcgacaccgcctgtgtgtgtgtgctgagcagaaaggctgtgagaagagcctacccgtacgacgtgccggactacgccaatgctgtgggccaggacacgcaggaggtcatcgtggtgccacactccttgccctttaaggtggtggtgatctcagccatcctggccctggtggtgctcaccatcatctcccttatcatcctcatcatgctttggcagaagaagccacgt

SEQ ID NO: 27 - RNA, 723 nt, synthetic construct
  misc_feature 1..723: note = BDNF19-NGFmut mRNA sequence
  source 1..723: mol_type = other RNA
atgaccatcctgttcctgacaatggtcattagctacttcggctgcatgaaggccgagcctcacagcgagtccaatgtgcccgccggccacaccatcccccaggctcattggaccaagctgcagcactccctggatacagccctgagaagagccagatccgcccctgccgccgccatcgctgctagagtggccggacagacaaggaacatcacagtggaccctagactgtttaagaagagaagactgaggagccccagagtgctgttctccacacagccccccagagaggccgccgacacacaggacctggatttcgaggtgggcggcgccgccccttttaacagaacacacagatccaagaggtccagctcccaccctatcttccacagaggcgagttcagcgtgtgcgactccgtgagcgtgtgggtgggcgacaagaccaccgccaccgacatcaagggcaaggaggtcatggtgctgggcgaggtgaacatcaataacagcgtgtttaagcagtactttttcgagaccaagtgcagagaccctaaccctgtggactccggctgtagaggcatcgactccaagcactggaatagctactgcaccacaacccacacattcgtgaaggccctgacaatggacggcaagcaggccgcctggtggttcatcagaatcgacaccgcctgtgtgtgtgtgctgagcagaaaggctgtgagaagagcc

SEQ ID NO: 28 - RNA, 744 nt, synthetic construct
  misc_feature 1..744: note = BDNF122-NGFmut mRNA sequence
  source 1..744: mol_type = other RNA
atgaccatcctgttcctgacaatggtcattagctacttcggctgcatgaaggccgcccccatgaaggaggccaacatcagaggccagggcggcctggcctaccccggagtgagaacacacggcacactggagtccgtgaacggccccaaggccggctccaggggactgacaagcctggccgataccttcgagcacgtgatcgaggagctgctggacgaggaccagaaggtgaggcccaatgaggagaataataaggacgccgacctgtacacctccagggtcatgctgagcagccaggtgcccctggagcccccactgctgttcctgctggaggagtacaagaattacctggacgccgccaatatgtccatgagagtgagaaggtccagctcccaccctatcttccacagaggcgagttcagcgtgtgcgactccgtgagcgtgtgggtgggcgacaagaccaccgccaccgacatcaagggcaaggaggtcatggtgctgggcgaggtgaacatcaataacagcgtgtttaagcagtactttttcgagaccaagtgcagagaccctaaccctgtggactccggctgtagaggcatcgactccaagcactggaatagctactgcaccacaacccacacattcgtgaaggccctgacaatggacggcaagcaggccgcctggtggttcatcagaatcgacaccgcctgtgtgtgtgtgctgagcagaaaggctgtgagaagagcc

SEQ ID NO: 29 - protein, 18 aa, synthetic construct
  REGION 1..18: note = NGF signal peptide
MSMLFYTLITAFLIGIQA

SEQ ID NO: 30 - protein, 21 aa, synthetic construct
  REGION 1..21: note = IGK signaling peptide
METDTLLLWVLLLWVPGSTGD

SEQ ID NO: 31 - protein, 18 aa, synthetic construct
  REGION 1..18: note = BDNF signaling peptide
MTILFLTMVISYFGCMKA

SEQ ID NO: 32 - protein, 120 aa, synthetic construct
  REGION 1..120: note = Matured NGFwt
SSSHPIFHRGEFSVCDSVSVWVGDKTTATDIKGKEVMVLGEVNINNSVFKQYFFETKCRDPNPVDSGCRGIDSKHWNSYCTTTHTFVKALTMDGKQAAWRFIRIDTACVCVLSRKAVRRA

SEQ ID NO: 33 - protein, 120 aa, synthetic construct
  REGION 1..120: note = Matured NGFmut
SSSHPIFHRGEFSVCDSVSVWVGDKTTATDIKGKEVMVLGEVNINNSVFKQYFFETKCRDPNPVDSGCRGIDSKHWNSYCTTTHTFVKALTMDGKQAAWWFIRIDTACVCVLSRKAVRRA

SEQ ID NO: 34 - protein, 103 aa, synthetic construct
  REGION 1..103: note = Leading peptide
EPHSESNVPAGHTIPQAHWTKLQHSLDTALRRARSAPAAAIAARVAGQTRNITVDPRLFKKRRLRSPRVLFSTQPPREAADTQDLDFEVGGAAPFNRTHRSKR

SEQ ID NO: 35 - protein, 20 aa, synthetic construct
  REGION 1..20: note = Human IL-2 signal peptide
MYRMQLLSCIALSLALVTNS

SEQ ID NO: 36 - protein, 19 aa, synthetic construct
  REGION 1..19: note = Azurin preprotein signal peptide
MTRLTVLALLAGLLASSRA

SEQ ID NO: 37 - protein, 19 aa, synthetic construct
  REGION 1..19: note = Immunoglobulin heavy chain signal peptide
MDWTWRVFCLLAVTPGAHP

SEQ ID NO: 38 - protein, 20 aa, synthetic construct
  REGION 1..20: note = Cystatin-S precursor signaling peptide
MARPLCTLLLLMATLAGALA

SEQ ID NO: 39 - protein, 38 aa, synthetic construct
  REGION 1..38: note = Gp67 signal peptide
MLLVNQSHQGFNKEHTSKMVSAIVLYVLLAAAAHSAFA

SEQ ID NO: 40 - protein, 21 aa, synthetic construct
  REGION 1..21: note = HBM signal peptides
MKFLVNVALVFMVVYISYIYA

SEQ ID NO: 41 - protein, 20 aa, synthetic construct
  REGION 1..20: note = Gp64 signal peptide
MVSAIVLYVLLAAAAHSAFA

SEQ ID NO: 42 - protein, 18 aa, synthetic construct
  REGION 1..18: note = Albumin signaling peptide
MKWVTFISLLFLFSSAYS

SEQ ID NO: 43 - protein, 26 aa, synthetic construct
  REGION 1..26: note = SP signal peptide
MRVTAPRTLILLLSGALALTETWAGS

SEQ ID NO: 44 - DNA, 1431 bp, synthetic construct
  misc_feature 1..1431: note = NGFwt-Fc1 optimized coding region sequence
  source 1..1431: mol_type = other DNA
atgtccatgttgttctacacactgatcaccgccttcctgatcggcatccaggccgagcctcacagcgagtccaatgtgcccgccggccacaccatcccccaggctcattggaccaagctgcagcactccctggatacagccctgagaagagccagatccgcccctgccgccgccatcgctgctagagtggccggacagacaaggaacatcacagtggaccctagactgtttaagaagagaagactgaggagccccagagtgctgttctccacacagccccccagagaggccgccgacacacaggacctggatttcgaggtgggcggcgccgccccttttaacagaacacacagatccaagaggtccagctcccaccctatcttccacagaggcgagttcagcgtgtgcgactccgtgagcgtgtgggtgggcgacaagaccaccgccaccgacatcaagggcaaggaggtcatggtgctgggcgaggtgaacatcaataacagcgtgtttaagcagtactttttcgagaccaagtgcagagaccctaaccctgtggactccggctgtagaggcatcgactccaagcactggaatagctactgcaccacaacccacacattcgtgaaggccctgacaatggacggcaagcaggccgcctggagattcatcagaatcgacaccgcctgtgtgtgtgtgctgagcagaaaggctgtgagaagagccggaagcggatctgagcccaagagccaggacaagacccacacctgccctccctgtcctgcccccgagctgctgggaggaccttccgtgttcctgttcccccctaagcctaaggacaccctgatgatcagcaggacccctgaggtgacctgtgtggtggtggatgtgtcccacgaggaccctgaggtgaagttcaactggtatgtggacggcgtggaggtgcacaatgctaagaccaagcccagggaagagcagtacaacagcacctatagggtggtgagcgtgctgaccgtgctgcaccaggattggctgaatggcaaggagtacaagtgtaaggtgagcaacaaggctctgcccgctcctatcgagaagaccatctccaaggccaagggccagcccagggagcctcaggtgtataccctgcccccttccagggatgagctgaccaagaatcaggtgtccctgacctgtctggtgaagggcttttacccctccgacatcgctgtggagtgggagagcaatggccagcctgagaacaactacaagaccacccctcccgtgctggattccgacggctcctttttcctgtatagcaagctgaccgtggacaagtcccggtggcagcagggcaacgtgttttcctgtagcgtgatgcacgaggccctgcacaaccactacacccagaagtccctgtccctgtcccctggcaag

SEQ ID NO: 45 - DNA, 1428 bp, synthetic construct
  misc_feature 1..1428: note = NGFwt-Fc2 optimized coding region sequence
  source 1..1428: mol_type = other DNA
atgtccatgttgttctacacactgatcaccgccttcctgatcggcatccaggccgagcctcacagcgagtccaatgtgcccgccggccacaccatcccccaggctcattggaccaagctgcagcactccctggatacagccctgagaagagccagatccgcccctgccgccgccatcgctgctagagtggccggacagacaaggaacatcacagtggaccctagactgtttaagaagagaagactgaggagccccagagtgctgttctccacacagccccccagagaggccgccgacacacaggacctggatttcgaggtgggcggcgccgccccttttaacagaacacacagatccaagaggtccagctcccaccctatcttccacagaggcgagttcagcgtgtgcgactccgtgagcgtgtgggtgggcgacaagaccaccgccaccgacatcaagggcaaggaggtcatggtgctgggcgaggtgaacatcaataacagcgtgtttaagcagtactttttcgagaccaagtgcagagaccctaaccctgtggactccggctgtagaggcatcgactccaagcactggaatagctactgcaccacaacccacacattcgtgaaggccctgacaatggacggcaagcaggccgcctggagattcatcagaatcgacaccgcctgtgtgtgtgtgctgagcagaaaggctgtgagaagagccggatctggcagcgataagaagatcgtgcccagggattgcggctgcaagccttgcatctgcaccgtgcccgaggtgtccagcgtgttcatcttcccccctaagcccaaggatgtgctgaccatcaccctgacccccaaggtgacctgtgtggtggtggctatcagcaaggatgaccccgaggtgcagtttagctggtttgtggatgatgtggaggtgcacaccgcccagacccagcccagagaggagcagtttaattccacctttcggtccgtgtccgagctgcctatcatgcaccaggattggctgaacggcaaggagttcaagtgcagggtgaacagcgctgctttccccgcccccatcgagaagaccatcagcaagaccaagggcaggcccaaggcccctcaggtgtacaccatccccccccccaaggagcagatggccaaggataaggtgagcctgacctgcatgatcaccgatttcttccctgaggatatcaccgtggagtggcagtggaatggccagcccgccgagaattacaagaatacccagcccatcatggacaccgacggcagctatttcgtgtattccaagctgaatgtgcagaagtccaattgggaggccggcaacaccttcacctgcagcgtgctgcacgagggcctgcacaatcaccacaccgagaagagcctgtcccactcccccggcaag

SEQ ID NO: 46 - DNA, 1440 bp, synthetic construct
  misc_feature 1..1440: note = IGK19-NGFmut-Fc1 optimized sequence of the coding region
  source 1..1440: mol_type = other DNA
atggagacagacacactcctgctatgggtactgctgctctgggttccaggttccaccggagacgagcctcacagcgagtccaatgtgcccgccggccacaccatcccccaggctcattggaccaagctgcagcactccctggatacagccctgagaagagccagatccgcccctgccgccgccatcgctgctagagtggccggacagacaaggaacatcacagtggaccctagactgtttaagaagagaagactgaggagccccagagtgctgttctccacacagccccccagagaggccgccgacacacaggacctggatttcgaggtgggcggcgccgccccttttaacagaacacacagatccaagaggtccagctcccaccctatcttccacagaggcgagttcagcgtgtgcgactccgtgagcgtgtgggtgggcgacaagaccaccgccaccgacatcaagggcaaggaggtcatggtgctgggcgaggtgaacatcaataacagcgtgtttaagcagtactttttcgagaccaagtgcagagaccctaaccctgtggactccggctgtagaggcatcgactccaagcactggaatagctactgcaccacaacccacacattcgtgaaggccctgacaatggacggcaagcaggccgcctggtggttcatcagaatcgacaccgcctgtgtgtgtgtgctgagcagaaaggctgtgagaagagccggaagcggatctgagcccaagagccaggacaagacccacacctgccctccctgtcctgcccccgagctgctgggaggaccttccgtgttcctgttcccccctaagcctaaggacaccctgatgatcagcaggacccctgaggtgacctgtgtggtggtggatgtgtcccacgaggaccctgaggtgaagttcaactggtatgtggacggcgtggaggtgcacaatgctaagaccaagcccagggaagagcagtacaacagcacctatagggtggtgagcgtgctgaccgtgctgcaccaggattggctgaatggcaaggagtacaagtgtaaggtgagcaacaaggctctgcccgctcctatcgagaagaccatctccaaggccaagggccagcccagggagcctcaggtgtataccctgcccccttccagggatgagctgaccaagaatcaggtgtccctgacctgtctggtgaagggcttttacccctccgacatcgctgtggagtgggagagcaatggccagcctgagaacaactacaagaccacccctcccgtgctggattccgacggctcctttttcctgtatagcaagctgaccgtggacaagtcccggtggcagcagggcaacgtgttttcctgtagcgtgatgcacgaggccctgcacaaccactacacccagaagtccctgtccctgtcccctggcaag

SEQ ID NO: 47 - DNA, 1437 bp, synthetic construct
  misc_feature 1..1437: note = IGK19-NGFmut-Fc2 optimized sequence of the coding region
  source 1..1437: mol_type = other DNA
atggagacagacacactcctgctatgggtactgctgctctgggttccaggttccaccggagacgagcctcacagcgagtccaatgtgcccgccggccacaccatcccccaggctcattggaccaagctgcagcactccctggatacagccctgagaagagccagatccgcccctgccgccgccatcgctgctagagtggccggacagacaaggaacatcacagtggaccctagactgtttaagaagagaagactgaggagccccagagtgctgttctccacacagccccccagagaggccgccgacacacaggacctggatttcgaggtgggcggcgccgccccttttaacagaacacacagatccaagaggtccagctcccaccctatcttccacagaggcgagttcagcgtgtgcgactccgtgagcgtgtgggtgggcgacaagaccaccgccaccgacatcaagggcaaggaggtcatggtgctgggcgaggtgaacatcaataacagcgtgtttaagcagtactttttcgagaccaagtgcagagaccctaaccctgtggactccggctgtagaggcatcgactccaagcactggaatagctactgcaccacaacccacacattcgtgaaggccctgacaatggacggcaagcaggccgcctggtggttcatcagaatcgacaccgcctgtgtgtgtgtgctgagcagaaaggctgtgagaagagccggatctggcagcgataagaagatcgtgcccagggattgcggctgcaagccttgcatctgcaccgtgcccgaggtgtccagcgtgttcatcttcccccctaagcccaaggatgtgctgaccatcaccctgacccccaaggtgacctgtgtggtggtggctatcagcaaggatgaccccgaggtgcagtttagctggtttgtggatgatgtggaggtgcacaccgcccagacccagcccagagaggagcagtttaattccacctttcggtccgtgtccgagctgcctatcatgcaccaggattggctgaacggcaaggagttcaagtgcagggtgaacagcgctgctttccccgcccccatcgagaagaccatcagcaagaccaagggcaggcccaaggcccctcaggtgtacaccatccccccccccaaggagcagatggccaaggataaggtgagcctgacctgcatgatcaccgatttcttccctgaggatatcaccgtggagtggcagtggaatggccagcccgccgagaattacaagaatacccagcccatcatggacaccgacggcagctatttcgtgtattccaagctgaatgtgcagaagtccaattgggaggccggcaacaccttcacctgcagcgtgctgcacgagggcctgcacaatcaccacaccgagaagagcctgtcccactcccccggcaag

SEQ ID NO: 48 - RNA, 1431 nt, synthetic construct
  misc_feature 1..1431: note = NGFwt-Fc1 mRNA Sequence
  source 1..1431: mol_type = other RNA
atgtccatgttgttctacacactgatcaccgccttcctgatcggcatccaggccgagcctcacagcgagtccaatgtgcccgccggccacaccatcccccaggctcattggaccaagctgcagcactccctggatacagccctgagaagagccagatccgcccctgccgccgccatcgctgctagagtggccggacagacaaggaacatcacagtggaccctagactgtttaagaagagaagactgaggagccccagagtgctgttctccacacagccccccagagaggccgccgacacacaggacctggatttcgaggtgggcggcgccgccccttttaacagaacacacagatccaagaggtccagctcccaccctatcttccacagaggcgagttcagcgtgtgcgactccgtgagcgtgtgggtgggcgacaagaccaccgccaccgacatcaagggcaaggaggtcatggtgctgggcgaggtgaacatcaataacagcgtgtttaagcagtactttttcgagaccaagtgcagagaccctaaccctgtggactccggctgtagaggcatcgactccaagcactggaatagctactgcaccacaacccacacattcgtgaaggccctgacaatggacggcaagcaggccgcctggagattcatcagaatcgacaccgcctgtgtgtgtgtgctgagcagaaaggctgtgagaagagccggaagcggatctgagcccaagagccaggacaagacccacacctgccctccctgtcctgcccccgagctgctgggaggaccttccgtgttcctgttcccccctaagcctaaggacaccctgatgatcagcaggacccctgaggtgacctgtgtggtggtggatgtgtcccacgaggaccctgaggtgaagttcaactggtatgtggacggcgtggaggtgcacaatgctaagaccaagcccagggaagagcagtacaacagcacctatagggtggtgagcgtgctgaccgtgctgcaccaggattggctgaatggcaaggagtacaagtgtaaggtgagcaacaaggctctgcccgctcctatcgagaagaccatctccaaggccaagggccagcccagggagcctcaggtgtataccctgcccccttccagggatgagctgaccaagaatcaggtgtccctgacctgtctggtgaagggcttttacccctccgacatcgctgtggagtgggagagcaatggccagcctgagaacaactacaagaccacccctcccgtgctggattccgacggctcctttttcctgtatagcaagctgaccgtggacaagtcccggtggcagcagggcaacgtgttttcctgtagcgtgatgcacgaggccctgcacaaccactacacccagaagtccctgtccctgtcccctggcaag

SEQ ID NO: 49 - RNA, 1428 nt, synthetic construct
  misc_feature 1..1428: note = NGFwt-Fc2 mRNA sequences
  source 1..1428: mol_type = other RNA
atgtccatgttgttctacacactgatcaccgccttcctgatcggcatccaggccgagcctcacagcgagtccaatgtgcccgccggccacaccatcccccaggctcattggaccaagctgcagcactccctggatacagccctgagaagagccagatccgcccctgccgccgccatcgctgctagagtggccggacagacaaggaacatcacagtggaccctagactgtttaagaagagaagactgaggagccccagagtgctgttctccacacagccccccagagaggccgccgacacacaggacctggatttcgaggtgggcggcgccgccccttttaacagaacacacagatccaagaggtccagctcccaccctatcttccacagaggcgagttcagcgtgtgcgactccgtgagcgtgtgggtgggcgacaagaccaccgccaccgacatcaagggcaaggaggtcatggtgctgggcgaggtgaacatcaataacagcgtgtttaagcagtactttttcgagaccaagtgcagagaccctaaccctgtggactccggctgtagaggcatcgactccaagcactggaatagctactgcaccacaacccacacattcgtgaaggccctgacaatggacggcaagcaggccgcctggagattcatcagaatcgacaccgcctgtgtgtgtgtgctgagcagaaaggctgtgagaagagccggatctggcagcgataagaagatcgtgcccagggattgcggctgcaagccttgcatctgcaccgtgcccgaggtgtccagcgtgttcatcttcccccctaagcccaaggatgtgctgaccatcaccctgacccccaaggtgacctgtgtggtggtggctatcagcaaggatgaccccgaggtgcagtttagctggtttgtggatgatgtggaggtgcacaccgcccagacccagcccagagaggagcagtttaattccacctttcggtccgtgtccgagctgcctatcatgcaccaggattggctgaacggcaaggagttcaagtgcagggtgaacagcgctgctttccccgcccccatcgagaagaccatcagcaagaccaagggcaggcccaaggcccctcaggtgtacaccatccccccccccaaggagcagatggccaaggataaggtgagcctgacctgcatgatcaccgatttcttccctgaggatatcaccgtggagtggcagtggaatggccagcccgccgagaattacaagaatacccagcccatcatggacaccgacggcagctatttcgtgtattccaagctgaatgtgcagaagtccaattgggaggccggcaacaccttcacctgcagcgtgctgcacgagggcctgcacaatcaccacaccgagaagagcctgtcccactcccccggcaag

SEQ ID NO: 50 - RNA, 1440 nt, synthetic construct
  misc_feature 1..1440: note = IGK19-NGFmut-Fc1 mRNA Sequence
  source 1..1440: mol_type = other RNA
atggagacagacacactcctgctatgggtactgctgctctgggttccaggttccaccggagacgagcctcacagcgagtccaatgtgcccgccggccacaccatcccccaggctcattggaccaagctgcagcactccctggatacagccctgagaagagccagatccgcccctgccgccgccatcgctgctagagtggccggacagacaaggaacatcacagtggaccctagactgtttaagaagagaagactgaggagccccagagtgctgttctccacacagccccccagagaggccgccgacacacaggacctggatttcgaggtgggcggcgccgccccttttaacagaacacacagatccaagaggtccagctcccaccctatcttccacagaggcgagttcagcgtgtgcgactccgtgagcgtgtgggtgggcgacaagaccaccgccaccgacatcaagggcaaggaggtcatggtgctgggcgaggtgaacatcaataacagcgtgtttaagcagtactttttcgagaccaagtgcagagaccctaaccctgtggactccggctgtagaggcatcgactccaagcactggaatagctactgcaccacaacccacacattcgtgaaggccctgacaatggacggcaagcaggccgcctggtggttcatcagaatcgacaccgcctgtgtgtgtgtgctgagcagaaaggctgtgagaagagccggaagcggatctgagcccaagagccaggacaagacccacacctgccctccctgtcctgcccccgagctgctgggaggaccttccgtgttcctgttcccccctaagcctaaggacaccctgatgatcagcaggacccctgaggtgacctgtgtggtggtggatgtgtcccacgaggaccctgaggtgaagttcaactggtatgtggacggcgtggaggtgcacaatgctaagaccaagcccagggaagagcagtacaacagcacctatagggtggtgagcgtgctgaccgtgctgcaccaggattggctgaatggcaaggagtacaagtgtaaggtgagcaacaaggctctgcccgctcctatcgagaagaccatctccaaggccaagggccagcccagggagcctcaggtgtataccctgcccccttccagggatgagctgaccaagaatcaggtgtccctgacctgtctggtgaagggcttttacccctccgacatcgctgtggagtgggagagcaatggccagcctgagaacaactacaagaccacccctcccgtgctggattccgacggctcctttttcctgtatagcaagctgaccgtggacaagtcccggtggcagcagggcaacgtgttttcctgtagcgtgatgcacgaggccctgcacaaccactacacccagaagtccctgtccctgtcccctggcaag

SEQ ID NO: 51 - RNA, 1437 nt, synthetic construct
  misc_feature 1..1437: note = IGK19-NGFmut-Fc2 mRNA Sequence
  source 1..1437: mol_type = other RNA
atggagacagacacactcctgctatgggtactgctgctctgggttccaggttccaccggagacgagcctcacagcgagtccaatgtgcccgccggccacaccatcccccaggctcattggaccaagctgcagcactccctggatacagccctgagaagagccagatccgcccctgccgccgccatcgctgctagagtggccggacagacaaggaacatcacagtggaccctagactgtttaagaagagaagactgaggagccccagagtgctgttctccacacagccccccagagaggccgccgacacacaggacctggatttcgaggtgggcggcgccgccccttttaacagaacacacagatccaagaggtccagctcccaccctatcttccacagaggcgagttcagcgtgtgcgactccgtgagcgtgtgggtgggcgacaagaccaccgccaccgacatcaagggcaaggaggtcatggtgctgggcgaggtgaacatcaataacagcgtgtttaagcagtactttttcgagaccaagtgcagagaccctaaccctgtggactccggctgtagaggcatcgactccaagcactggaatagctactgcaccacaacccacacattcgtgaaggccctgacaatggacggcaagcaggccgcctggtggttcatcagaatcgacaccgcctgtgtgtgtgtgctgagcagaaaggctgtgagaagagccggatctggcagcgataagaagatcgtgcccagggattgcggctgcaagccttgcatctgcaccgtgcccgaggtgtccagcgtgttcatcttcccccctaagcccaaggatgtgctgaccatcaccctgacccccaaggtgacctgtgtggtggtggctatcagcaaggatgaccccgaggtgcagtttagctggtttgtggatgatgtggaggtgcacaccgcccagacccagcccagagaggagcagtttaattccacctttcggtccgtgtccgagctgcctatcatgcaccaggattggctgaacggcaaggagttcaagtgcagggtgaacagcgctgctttccccgcccccatcgagaagaccatcagcaagaccaagggcaggcccaaggcccctcaggtgtacaccatccccccccccaaggagcagatggccaaggataaggtgagcctgacctgcatgatcaccgatttcttccctgaggatatcaccgtggagtggcagtggaatggccagcccgccgagaattacaagaatacccagcccatcatggacaccgacggcagctatttcgtgtattccaagctgaatgtgcagaagtccaattgggaggccggcaacaccttcacctgcagcgtgctgcacgagggcctgcacaatcaccacaccgagaagagcctgtcccactcccccggcaag

SEQ ID NO: 52 - protein, 477 aa, synthetic construct
  REGION 1..477: note = NGFwt-Fc1 amino acid sequence
MSMLFYTLITAFLIGIQAEPHSESNVPAGHTIPQAHWTKLQHSLDTALRRARSAPAAAIAARVAGQTRNITVDPRLFKKRRLRSPRVLFSTQPPREAADTQDLDFEVGGAAPFNRTHRSKRSSSHPIFHRGEFSVCDSVSVWVGDKTTATDIKGKEVMVLGEVNINNSVFKQYFFETKCRDPNPVDSGCRGIDSKHWNSYCTTTHTFVKALTMDGKQAAWRFIRIDTACVCVLSRKAVRRAGSGSEPKSQDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 53 - protein, 476 aa, synthetic construct
  REGION 1..476: note = NGFwt-Fc2 amino acid sequence
MSMLFYTLITAFLIGIQAEPHSESNVPAGHTIPQAHWTKLQHSLDTALRRARSAPAAAIAARVAGQTRNITVDPRLFKKRRLRSPRVLFSTQPPREAADTQDLDFEVGGAAPFNRTHRSKRSSSHPIFHRGEFSVCDSVSVWVGDKTTATDIKGKEVMVLGEVNINNSVFKQYFFETKCRDPNPVDSGCRGIDSKHWNSYCTTTHTFVKALTMDGKQAAWRFIRIDTACVCVLSRKAVRRAGSGSDKKIVPRDCGCKPCICTVPEVSSVFIFPPKPKDVLTITLTPKVTCVVVAISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 54 - protein, 480 aa, synthetic construct
  REGION 1..480: note = IGK19-NGFmut-Fc1 amino acid sequence
METDTLLLWVLLLWVPGSTGDEPHSESNVPAGHTIPQAHWTKLQHSLDTALRRARSAPAAAIAARVAGQTRNITVDPRLFKKRRLRSPRVLFSTQPPREAADTQDLDFEVGGAAPFNRTHRSKRSSSHPIFHRGEFSVCDSVSVWVGDKTTATDIKGKEVMVLGEVNINNSVFKQYFFETKCRDPNPVDSGCRGIDSKHWNSYCTTTHTFVKALTMDGKQAAWWFIRIDTACVCVLSRKAVRRAGSGSEPKSQDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 55 - protein, 479 aa, synthetic construct
  REGION 1..479: note = IGK19-NGFmut-Fc2 amino acid sequence
METDTLLLWVLLLWVPGSTGDEPHSESNVPAGHTIPQAHWTKLQHSLDTALRRARSAPAAAIAARVAGQTRNITVDPRLFKKRRLRSPRVLFSTQPPREAADTQDLDFEVGGAAPFNRTHRSKRSSSHPIFHRGEFSVCDSVSVWVGDKTTATDIKGKEVMVLGEVNINNSVFKQYFFETKCRDPNPVDSGCRGIDSKHWNSYCTTTHTFVKALTMDGKQAAWWFIRIDTACVCVLSRKAVRRAGSGSDKKIVPRDCGCKPCICTVPEVSSVFIFPPKPKDVLTITLTPKVTCVVVAISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 56 - protein, 232 aa, synthetic construct
  REGION 1..232: note = Human Fc-1 amino acid sequence
EPKSQDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 57 - protein, 231 aa, synthetic construct
  REGION 1..231: note = Mouse Fc-2 amino acid sequence
DKKIVPRDCGCKPCICTVPEVSSVFIFPPKPKDVLTITLTPKVTCVVVAISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 58 - protein, 249 aa, synthetic construct
  REGION 1..249: note = SP-19-NGFmut amino acid sequence
MRVTAPRTLILLLSGALALTETWAGSEPHSESNVPAGHTIPQAHWTKLQHSLDTALRRARSAPAAAIAARVAGQTRNITVDPRLFKKRRLRSPRVLFSTQPPREAADTQDLDFEVGGAAPFNRTHRSKRSSSHPIFHRGEFSVCDSVSVWVGDKTTATDIKGKEVMVLGEVNINNSVFKQYFFETKCRDPNPVDSGCRGIDSKHWNSYCTTTHTFVKALTMDGKQAAWWFIRIDTACVCVLSRKAVRRA

SEQ ID NO: 59 - protein, 243 aa, synthetic construct
  REGION 1..243: note = hIL12-19-NGFmut amino acid sequence
MYRMQLLSCIALSLALVTNSEPHSESNVPAGHTIPQAHWTKLQHSLDTALRRARSAPAAAIAARVAGQTRNITVDPRLFKKRRLRSPRVLFSTQPPREAADTQDLDFEVGGAAPFNRTHRSKRSSSHPIFHRGEFSVCDSVSVWVGDKTTATDIKGKEVMVLGEVNINNSVFKQYFFETKCRDPNPVDSGCRGIDSKHWNSYCTTTHTFVKALTMDGKQAAWWFIRIDTACVCVLSRKAVRRA

SEQ ID NO: 60 - protein, 243 aa, synthetic construct
  REGION 1..243: note = Cystatin 19-NGFmut amino acid sequence
MARPLCTLLLLMATLAGALAEPHSESNVPAGHTIPQAHWTKLQHSLDTALRRARSAPAAAIAARVAGQTRNITVDPRLFKKRRLRSPRVLFSTQPPREAADTQDLDFEVGGAAPFNRTHRSKRSSSHPIFHRGEFSVCDSVSVWVGDKTTATDIKGKEVMVLGEVNINNSVFKQYFFETKCRDPNPVDSGCRGIDSKHWNSYCTTTHTFVKALTMDGKQAAWWFIRIDTACVCVLSRKAVRRA

SEQ ID NO: 61 - protein, 242 aa, synthetic construct
  REGION 1..242: note = APP-19-NGFmut amino acid sequence
MTRLTVLALLAGLLASSRAEPHSESNVPAGHTIPQAHWTKLQHSLDTALRRARSAPAAAIAARVAGQTRNITVDPRLFKKRRLRSPRVLFSTQPPREAADTQDLDFEVGGAAPFNRTHRSKRSSSHPIFHRGEFSVCDSVSVWVGDKTTATDIKGKEVMVLGEVNINNSVFKQYFFETKCRDPNPVDSGCRGIDSKHWNSYCTTTHTFVKALTMDGKQAAWWFIRIDTACVCVLSRKAVRRA

SEQ ID NO: 62 - protein, 242 aa, synthetic construct
  REGION 1..242: note = HC-19-NGFmut amino acid sequence
MDWTWRVFCLLAVTPGAHPEPHSESNVPAGHTIPQAHWTKLQHSLDTALRRARSAPAAAIAARVAGQTRNITVDPRLFKKRRLRSPRVLFSTQPPREAADTQDLDFEVGGAAPFNRTHRSKRSSSHPIFHRGEFSVCDSVSVWVGDKTTATDIKGKEVMVLGEVNINNSVFKQYFFETKCRDPNPVDSGCRGIDSKHWNSYCTTTHTFVKALTMDGKQAAWWFIRIDTACVCVLSRKAVRRA

SEQ ID NO: 63 - protein, 261 aa, synthetic construct
  REGION 1..261: note = Gp67-19-NGFmut amino acid sequence
MLLVNQSHQGFNKEHTSKMVSAIVLYVLLAAAAHSAFAEPHSESNVPAGHTIPQAHWTKLQHSLDTALRRARSAPAAAIAARVAGQTRNITVDPRLFKKRRLRSPRVLFSTQPPREAADTQDLDFEVGGAAPFNRTHRSKRSSSHPIFHRGEFSVCDSVSVWVGDKTTATDIKGKEVMVLGEVNINNSVFKQYFFETKCRDPNPVDSGCRGIDSKHWNSYCTTTHTFVKALTMDGKQAAWWFIRIDTACVCVLSRKAVRRA

SEQ ID NO: 64 - protein, 244 aa, synthetic construct
  REGION 1..244: note = Amino acid sequence of HBM-19-NGFmut
MKFLVNVALVFMVVYISYIYAEPHSESNVPAGHTIPQAHWTKLQHSLDTALRRARSAPAAAIAARVAGQTRNITVDPRLFKKRRLRSPRVLFSTQPPREAADTQDLDFEVGGAAPFNRTHRSKRSSSHPIFHRGEFSVCDSVSVWVGDKTTATDIKGKEVMVLGEVNINNSVFKQYFFETKCRDPNPVDSGCRGIDSKHWNSYCTTTHTFVKALTMDGKQAAWWFIRIDTACVCVLSRKAVRRA

SEQ ID NO: 65 - protein, 243 aa, synthetic construct
  REGION 1..243: note = Gp64-19-NGFmut amino acid sequence
MVSAIVLYVLLAAAAHSAFAEPHSESNVPAGHTIPQAHWTKLQHSLDTALRRARSAPAAAIAARVAGQTRNITVDPRLFKKRRLRSPRVLFSTQPPREAADTQDLDFEVGGAAPFNRTHRSKRSSSHPIFHRGEFSVCDSVSVWVGDKTTATDIKGKEVMVLGEVNINNSVFKQYFFETKCRDPNPVDSGCRGIDSKHWNSYCTTTHTFVKALTMDGKQAAWWFIRIDTACVCVLSRKAVRRA

SEQ ID NO: 66 - protein, 241 aa, synthetic construct
  REGION 1..241: note = Alb-19-NGFmut amino acid sequence
MKWVTFISLLFLFSSAYSEPHSESNVPAGHTIPQAHWTKLQHSLDTALRRARSAPAAAIAARVAGQTRNITVDPRLFKKRRLRSPRVLFSTQPPREAADTQDLDFEVGGAAPFNRTHRSKRSSSHPIFHRGEFSVCDSVSVWVGDKTTATDIKGKEVMVLGEVNINNSVFKQYFFETKCRDPNPVDSGCRGIDSKHWNSYCTTTHTFVKALTMDGKQAAWWFIRIDTACVCVLSRKAVRRA

SEQ ID NO: 67 - protein, 50 aa, synthetic construct
  REGION 1..50: note = PDGFR
NAVGQDTQEVIVVPHSLPFKVVVISAILALVVLTIISLIILIMLWQKKPR

SEQ ID NO: 68 - protein, 9 aa, synthetic construct
  REGION 1..9: note = Label Peptide 1st
YPYDVPDYA

SEQ ID NO: 69 - protein, 18 aa, synthetic construct
  REGION 1..18: note = Label Peptide 2nd
YPYDVPDYAYPYDVPDYA

SEQ ID NO: 70 - protein, 253 aa, synthetic construct; identical to SEQ ID NO: 15
  REGION 1..253: note = IGK19-NGFmut amino acid sequence (with label)

(19) United States
(12) Patent Application Publication
(10) Pub. No.: US 2025/0009846 A1
(43) Pub. Date: Jan. 9, 2025

(54) RECOMBINANT PROTEIN OF NERVE GROWTH FACTOR MUTANT AND USE THEREOF

(71) Applicants: SHANGHAI JIAO TONG UNIVERSITY SCHOOL OF MEDICINE, Shanghai (CN); SHANGHAI RNACURE BIOPHARMA CO., LTD., Shanghai (CN)

(72) Inventors: Yingjie XU, Shanghai (CN); Xiang YU, Shanghai (CN); Wei XU, Shanghai (CN); Wen YANG, Shanghai (CN); Zheng YANG, Shanghai (CN); Yu ZHANG, Shanghai (CN)

(21) Appl. No.: 18/686,041

(22) PCT Filed: Aug. 24, 2022
(86) PCT No.: PCT/CN2022/114516

§ 371 Date: Feb. 23, 2024

(30) Foreign Application Priority Data
Aug. 25, 2021 (CN) 202110981721.5

(51) Int. Cl. A61K 38/18; A61K 9/127; A61P 25/02; C07K 14/48

(52) U.S. Cl. CPC A61K 38/185; A61K 9/127; A61P 25/02; C07K 14/48

(57) ABSTRACT

Provided is a recombinant protein, which contains a nerve growth factor (NGF) protein variant and a heterologous signal peptide linked to the NGF protein variant. Further provided are a combination containing an mRNA encoding the recombinant protein and a delivery vector, and the use thereof in the treatment of toxic/hereditary/metabolic peripheral neuropathy, nerve regeneration repair, and/or central nervous system degenerative diseases.

### TECHNICAL FIELD

The present application relates to the field of biological medicines, and particularly relates to a recombinant protein of a nerve growth factor mutant, including an mRNA encoding the recombinant protein, as well as a use thereof in treatment of nervous system diseases.

### BACKGROUND

A nerve growth factor (NGF) is a growth regulatory factor with a neuronal nutritional function, playing an important role in maintaining the development and functional differentiation of central basal forebrain cholinergic neurons (BFCNs) and peripheral nociceptive sensory neurons. It is also crucial for maintaining the normal functions of mature central and peripheral neurons, as well as for self-protection and repair of nerve injuries. The NGF, as a powerful neurotrophic factor, has become a hot topic in drug therapy for various central and peripheral nervous system diseases in the past 20-30 years.

In the process of clinical research, the application of the NGF has led to dose-dependent pain side effects. Alzheimer's disease (AD) is a progressive and fatal central nervous system degenerative disease characterized by cognitive impairment and memory impairment. In clinical experiments, high-dose NGFs are used to treat AD patients, and it is found that although the cognitive ability and cortical metabolism of the AD patients are significantly improved, the occurrence of adverse reactions such as pain and weight loss offsets their therapeutic effect, ultimately declaring the failure of the clinical experiments. The same phenomenon is also found in the treatment of diabetic neuropathy and HIV peripheral neuropathy with the NGF. This is because the NGF is one of the important mediators that cause pain and hyperalgesia. Both TrkA and p75NTR signaling pathways mediate the pain inducing function of the NGF, and many studies have shown that the TrkA and p75NTR signaling pathways interact with each other. Therefore, losing any signaling pathway (TrkA or p75NTR) or breaking the balance between the TrkA and p75NTR signaling pathways will have an impact on the pain-inducing effect of the NGF.

The discovery of “painless” nerve growth factors has brought a great hope for the clinical application of the NGF. A hereditary sensory and autonomous neuropathy type V (HSAN V) secondary to an NGF^{R100W} is a mature NGF protein found in a northern Swedish family with arginine at the 100^{th} position being replaced with tryptophan, patients experience loss of pain and temperature perception, but their intelligence is normal, suggesting that the NGF^{R100W} may selectively lose the function of mediating pain, while retaining the neurotrophic function. The discovery of the NGF^{R100W} provides an important basis for studying the neurotrophic and pain mediated functions of the NGF, and the study of its biological characteristics and related mechanisms of action may seek new breakthroughs in the treatment of toxic/hereditary/metabolic peripheral neuropathy, nerve regeneration repair, and/or central nervous system degenerative diseases using the NGF.

However, a knock-in mouse model of the HSAN V demonstrates that mice expressing an NGF^{R100W} mutant typically completely lose pain at approximately 2 months of age and are often unable to survive into adulthood. Besides, a full-length NGF^{R100W} mutant protein can affect the cleavage of its mature body, thereby preventing the normal secretion of a mature NGF^{R100W} into the extracellular space to exercise its neurotrophic function. Therefore, there is currently no simple and efficient method to obtain the NGF^{R100W} mutant protein, greatly limiting the application of the NGF^{R100W} mutant protein in the treatment of nerve diseases.

### SUMMARY

The present application provides a recombinant protein, and the recombinant protein includes a nerve growth factor (NGF) protein variant and a heterologous signal peptide linked to the NGF protein variant. The recombinant protein has at least one of the following characteristics: (1) compared with a wild type NGF protein, binding of the NGF protein variant to a neurotrophin receptor p75 (p75NTR) is reduced; (2) an expression amount and/or a secretion amount are/is increased by at least 50%; (3) it has a considerable expression amount and/or secretion amount, for example, the expression amount and/or the secretion amount may reach at least 50% or above of those of the wild type NGF protein; (4) pain is reduced; (5) it has a prolonged half-life period; and (6) repair and regeneration of injured nerve cells are promoted. The present application further provides a composition including an mRNA encoding an NGF protein or the NGF protein variant and a delivery vector. The recombinant protein and the composition of the present application can be used for treating nervous system diseases such as peripheral neuropathy and neurodegenerative diseases and promoting nerve repair and regeneration. For example, the recombinant protein and the composition of the present application can be used for treating toxic/hereditary/metabolic peripheral neuropathy, nerve regeneration repair and/or central nervous system degenerative diseases (such as diabetic peripheral neuropathy, drug-related peripheral neuropathy, hereditary motor sensory neuropathy, post-traumatic repair of peripheral nerves, traumatic optic neuritis and/or Alzheimer's disease).

In one aspect, the present application provides a recombinant protein, and the recombinant protein includes a nerve growth factor (NGF) protein variant and a heterologous signal peptide linked to the NGF protein variant, wherein compared with a wild type NGF protein, binding of the NGF protein variant to a neurotrophin receptor p75 (p75NTR) is reduced by at least about 50%, and compared with a signal peptide shown in SEQ ID NO: 29, the heterologous signal peptide increases an expression amount and/or a secretion amount of the NGF protein variant by at least about 50%.

In certain implementations, the wild type NGF protein contains an amino acid sequence shown in SEQ ID NO: 32.

In certain implementations, the NGF protein variant contains an amino acid sequence shown in SEQ ID NO: 33.

In certain implementations, the heterologous signal peptide is derived from a brain-derived neurotrophic factor (BDNF), a neurotrophin-3 (NT-3) and/or a neurotrophin-4 (NT-4).

In certain implementations, the heterologous signal peptide is derived from Gp67, Gp64, honeybee melittin (HBM), albumin, IL-2, azurocidin preproprotein, mouse IgK, an immunoglobulin heavy chain and/or a cystatin-S precursor.

In certain implementations, the recombinant protein further includes a leader peptide.

In certain implementations, from an N terminal to a C terminal, the recombinant protein sequentially includes the heterologous signal peptide, the leader peptide and the NGF protein variant.

In certain implementations, the recombinant protein further includes an Fc region of immunoglobulin.

In certain implementations, from the N terminal to the C terminal, the recombinant protein sequentially includes the heterologous signal peptide, the leader peptide, the NGF protein variant and the Fc region.

In another aspect, the present application provides an isolated nucleic acid molecule, and the isolated nucleic acid molecule encodes the recombinant protein.

In certain implementations, the isolated nucleic acid molecule includes a DNA and/or an RNA. In certain implementations, the isolated nucleic acid molecule is a DNA. In certain implementations, the isolated nucleic acid molecule is an RNA.

In certain implementations, the isolated nucleic acid molecule contains modifications at one or more positions selected from: a 5′ cap, a 5′ untranslated region, an open reading frame, a 3′ untranslated region, and a poly A tail.

In certain implementations, the isolated nucleic acid molecule contains at least one modified nucleotide.

In certain implementations, the modified nucleotide contained in the isolated nucleic acid molecule contains one or more nucleotides selected from: N1-methylpseudouridine-5′-triphosphate (N1-Methylpseudo-UTP), pseudouridine-5′-triphosphate (pseudo-UTP), 5-methoxyuridine-5′-triphosphate (5-Methoxy-UTP) and 5-methylcytidine-5′-triphosphate (5-Methyl-CTP).

In certain implementations, the isolated nucleic acid molecule is codon-optimized.

In another aspect, the present application provides a composition, including (a) an mRNA, containing a polynucleotide encoding a nerve growth factor (NGF) protein or a variant thereof, and (b) a delivery vector.

In certain implementations, the NGF protein contains an amino acid sequence shown in SEQ ID NO: 32.

In certain implementations, compared with the amino acid sequence shown in SEQ ID NO: 32, binding of the NGF protein variant to a neurotrophin receptor p75 (p75NTR) is reduced by at least about 50%.

In certain implementations, the NGF protein variant contains an amino acid sequence shown in SEQ ID NO: 33.

In certain implementations, the mRNA contains a polynucleotide encoding a heterologous signal peptide, and compared with a signal peptide shown in SEQ ID NO: 29, the heterologous signal peptide increases an expression amount and/or a secretion amount of the NGF protein variant by at least about 50%.

In certain implementations, the heterologous signal peptide enables the expression amount and/or the secretion amount of the NGF protein variant to reach 50% or above of those of a wild type NGF protein.

In certain implementations, the heterologous signal peptide is derived from a brain-derived neurotrophic factor (BDNF), a neurotrophin-3 (NT-3) and/or a neurotrophin-4 (NT-4).

In certain implementations, the heterologous signal peptide is derived from Gp67, Gp64, honeybee melittin (HBM), albumin, IL-2, azurocidin preproprotein, mouse IgK, an immunoglobulin heavy chain and/or a cystatin-S precursor.

In certain implementations, the mRNA further includes a polynucleotide encoding a leader peptide.

In certain implementations, from a 5′ terminal to a 3′ terminal, the mRNA sequentially includes the polynucleotide encoding the heterologous signal peptide, the polynucleotide encoding the leader peptide, and the polynucleotide encoding the NGF protein or the variant thereof.

In certain implementations, from the 5′ terminal to the 3′ terminal, the mRNA sequentially includes a polynucleotide encoding the IgK signal peptide, the polynucleotide encoding the leader peptide, and the polynucleotide encoding the NGF protein variant.

In certain implementations, from the 5′ terminal to the 3′ terminal, the mRNA sequentially includes a polynucleotide encoding the BDNF signal peptide, the polynucleotide encoding the leader peptide, and the polynucleotide encoding the NGF protein variant.

In certain implementations, the mRNA further contains a polynucleotide encoding an Fc region of immunoglobulin.

In certain implementations, from the 5′ terminal to the 3′ terminal, the mRNA sequentially includes the polynucleotide encoding the heterologous signal peptide, the polynucleotide encoding the leader peptide, the polynucleotide encoding the NGF protein variant, and the polynucleotide encoding the Fc region of the immunoglobulin.

In certain implementations, from the 5′ terminal to the 3′ terminal, the mRNA sequentially includes the polynucleotide encoding the IgK signal peptide, the polynucleotide encoding the leader peptide, the polynucleotide encoding the NGF protein variant, and the polynucleotide encoding the Fc region of the immunoglobulin.

In certain implementations, the mRNA encodes and contains an amino acid sequence shown in any one of SEQ ID NOs: 54-55.

In certain implementations, the mRNA contains modifications at one or more positions selected from: a 5′ cap, a 5′ untranslated region, an open reading frame, a 3′ untranslated region, and a poly A tail.

In certain implementations, the mRNA contains at least one modified nucleotide.

In certain implementations, the modified nucleotide contained in the mRNA contains one or more nucleotides selected from: N1-methylpseudouridine-5′-triphosphate (N1-Methylpseudo-UTP), pseudouridine-5′-triphosphate (pseudo-UTP), 5-methoxyuridine-5′-triphosphate (5-Methoxy-UTP) and 5-methylcytidine-5′-triphosphate (5-Methyl-CTP).

In certain implementations, the mRNA is codon-optimized.

In certain implementations, the mRNA contains a nucleotide sequence shown in any one of SEQ ID NOs: 22-28 and 50-51. In certain implementations, the mRNA contains a nucleotide sequence shown in any one of SEQ ID NOs: 23 and 27.

In certain implementations, the delivery vector includes liposome.

In certain implementations, the delivery vector includes lipid nanoparticles (LNPs).

In certain implementations, the delivery vector includes cationic lipid.

In certain implementations, a molar ratio of the cationic lipid is about 45% to about 55%.

In certain implementations, the cationic lipid is SM102 and DLin-MC3-DMA.

In certain implementations, the delivery vector includes non-cationic lipid.

In certain implementations, the non-cationic lipid includes phospholipid and/or a lipid conjugate.

In certain implementations, a molar ratio of the phospholipid is about 35% to about 40%.

In certain implementations, the phospholipid includes distearyl phosphatidylcholine (DSPC).

In certain implementations, the lipid conjugate includes a polyethylene glycol-modified lipid molecule.

In certain implementations, in the delivery vector, a molar ratio of the lipid conjugate is about 1% to about 2%.

In certain implementations, the polyethylene glycol-modified lipid molecule includes PEG2000-DMG.

In certain implementations, the delivery vector includes cholesterol.

In certain implementations, in the delivery vector, a molar ratio of the cholesterol is about 8% to about 12%.

In certain implementations, the delivery vector contains the cationic lipid, the cholesterol, the phospholipid, and the lipid conjugate, and a mass ratio of the cationic lipid to the cholesterol to the phospholipid to the lipid conjugate is 50:10:38.5:1.5.

In certain implementations, the delivery vector contains the DLin-MC3-DMA, the cholesterol, the DSPC, and PEG2000-DMPE, and a mass ratio of the DLin-MC3-DMA to the cholesterol to the DSPC to the PEG2000-DMPE is 50:10:38.5:1.5.

In certain implementations, the delivery vector has a diameter of about 60 nm to about 500 nm.

In certain implementations, the delivery vector has a diameter of about 80 nm to about 200 nm.

In certain implementations, the mRNA is encapsulated in the delivery vector.

In another aspect, the present application provides a vector, the vector including the nucleic acid molecule.

In another aspect, the present application provides a cell, the cell including the nucleic acid molecule and/or the vector of the present application, or expressing the recombinant protein of the present application.

In another aspect, the present application provides a method for preparing the recombinant protein, and the method includes culturing the cell under a condition of expressing the recombinant protein.

In another aspect, the present application provides a pharmaceutical composition, and the pharmaceutical composition includes the recombinant protein, the nucleic acid molecule, the composition, the vector and/or the cell, and optionally a pharmaceutically acceptable carrier.

In another aspect, the present application provides a kit or a drug administration device, and the kit or the drug administration device includes the recombinant protein, the nucleic acid molecule, the composition, the vector, the cell, and/or the pharmaceutical composition.

In another aspect, the present application provides a use of the recombinant protein, the nucleic acid molecule, the composition, the vector, the cell and/or the pharmaceutical composition in preparation of a medication for relieving, preventing and/or treating central and/or peripheral nervous system diseases.

Those skilled in the art can easily gain insight into other aspects and advantages of the present application from the detailed description below. The detailed description below only shows and describes exemplary implementations of the present application. As those skilled in the art will recognize, the content of the present application enables those skilled in the art to make modifications to the disclosed specific implementations without departing from the spirit and scope of the invention referred to in the present application. Correspondingly, the descriptions in the accompanying drawings and specification of the present application are only illustrative and not restrictive.

### BRIEF DESCRIPTION OF THE DRAWINGS

The specific features of the invention referred to in the present application are shown in the attached claims. By referring to the exemplary implementations and accompanying drawings described in detail below, the characteristics and advantages of the invention referred to in the present application can be better understood. A brief explanation of the accompanying drawings is as follows:

FIG. 1A shows a plurality of compositions of an NGF^{R100W} recombinant protein.

FIG. 1B shows expression situations of different NGF^{R100W} recombinant proteins in a 293T cell.

FIG. 1C shows an expression situation of an IGK19-NGFmut mRNA containing different nucleotide modifications.

FIG. 1D shows fluorescence microscope observation results of expressing and promoting PC12 cell differentiation of NGF and NGF^{R100W} recombinant protein mRNAs in a PC12 cell.

FIG. 1E shows electron microscope observation results of promoting PC12 cell differentiation of NGF and NGF^{R100W} recombinant protein mRNAs.

FIG. 1F to FIG. 1G show detection results of contents of a mature NGF protein and a mature NGF^{R100W} protein in a cell lysis solution and a cell supernatant.

FIG. 1H to FIG. 1I show detection results of contents of a mature NGF protein and a mature NGF^{R100W} protein in a cell supernatant.

FIG. 1J shows a plurality of compositions of an NGF^{R100W} recombinant protein.

FIG. 2 shows assembly and in-vivo delivery of mRNA-LNPs.

FIG. 3A shows a nano particle size distribution of NGF and NGF^{R100W} recombinant protein mRNAs.

FIG. 3B shows expression situations of NGF and NGF^{R100W} recombinant protein mRNAs in a 293T cell.

FIG. 3C shows in-vivo expression positioning of IGK19-NGFmut mRNA LNP mice.

FIG. 4A shows an impact of expression of an NGF and IGK19-NGFmut on a pain threshold of mouse heat pain.

FIG. 4B shows an impact of expression of an NGF and IGK19-NGFmut on a pain threshold of mouse mechanical pain.

FIG. 5A shows an experimental process of expression of IGK19-NGFmut on peripheral nerve injury repair in mice.

FIG. 5B shows an impact of injection of paclitaxel on pain thresholds of mouse heat pain and mechanical pain.

FIG. 5C shows a repair situation of expression of IGK19-NGFmut on injured peripheral nerves in mice.

FIG. 5D shows an impact of expression of IGK19-NGFmut on pain thresholds of heat pain and mechanical pain of mice with peripheral nerve injuries.

### DETAILED DESCRIPTION

The implementations of the present application are described below by particular specific examples, and those skilled in the art can easily understand other advantages and effects of the present application from the contents disclosed in the specification.

### Term Definitions

In the present application, the term “nerve growth factor (NGF)” generally refers to a secretory protein that works in growth and survival of various neurons. As used herein, the term NGF is not limited to a human NGF, and contains all species orthologs of the human NGF. The term “NGF” covers a pro-form of the NGF, a proNGF, a full-length NGF and any form of NGF produced in an intracellular process. The term also covers NGF variants that are present naturally, such as a splicing variant, an allelic variant and an isotype.

The NGF is present in a complex of about 130-kDa composed of three proteins α-NGF, β-NGF and γ-NGF, and such NGF is also called a proNGF. The proNGF is also called an immature NGF. A γ subunit of the complex plays a role of serine protease, and can lyse an n terminal of a β subunit, thereby activating the protein to generate an NGF with functions, namely β-NGF. The NGF can bind to two receptors: a p75 neurotrophin receptor (p75NTR) and TrkA, which are transmembrane tyrosine kinases. The NGF is a fully validated target for pain, known to mediate the sensitization effect of nociceptors. An amino acid sequence of a human full-length NGF can be found in the Genbank login number NP_002497.2 or the UniProtKB login number P01138. The human full-length NGF typically contains 241 amino acids, with positions 1-18 being NGF signal peptide portions (an amino acid sequence as shown in SEQ ID NO: 29), positions 19-121 being leader peptide portions (an amino acid sequence as shown in SEQ ID NO: 34), and positions 122-241 being mature NGFs (an amino acid sequence as shown in SEQ ID NO: 32). The full-length NGF may become the mature NGF through a shearing process.

In the present application, the term “variant” generally refers to, compared to a reference sequence, an amino acid sequence that has one or more amino acids changed (such as substitution, variation, modification, replacement, deletion or addition of one or more amino acids), or a nucleic acid sequence that has one or more nucleotides changed (such as substitution, variation, modification, replacement, deletion or addition of one or more nucleotides). For polypeptides, a variant includes a polypeptide with an altered amino acid sequence due to substitution, deletion, or insertion of one or more amino acids. A variant may be present naturally or non-naturally. A variant may retain one or more activities of a reference sequence, but compared to the reference sequence, the one or more activities of the variant may be enhanced, weakened, or maintained unchanged.

In the present application, the term “NGF variant” generally refers to, compared to a full-length NGF amino acid sequence (for example, the Genbank login number NP_002497.2 or the UniProtKB login number P01138, or an amino acid sequence as shown in SEQ ID NO: 13), an amino acid sequence that has one or more amino acids changed (such as substitution, variation, modification, replacement, deletion or addition of one or more amino acids). For example, the amino acids may change in a mature NGF portion. Compared to a wild type NGF protein, the change of the amino acids (such as mutation) makes binding of an NGF protein variant to a neurotrophin receptor p75 (p75NTR) be reduced. For example, for a full-length NGF sequence with an amino acid sequence as shown in SEQ ID NO: 13, amino acid mutation may be mutation of arginine (R) at the position 221 to tryptophan (W), and the mutated full-length NGF protein may be called an NGF^{R221W} (an amino acid sequence as shown in SEQ ID NO: 14). For example, for a mature NGF sequence with an amino acid sequence as shown in SEQ ID NO: 32, amino acid mutation may be mutation of arginine (R) at the position 100 to tryptophan (W), and the mutated mature NGF protein may be called an NGF^{R100W} (an amino acid sequence as shown in SEQ ID NO: 33).

In the present application, the term “link” may be used interchangeably with “fused” or “fusion”, and generally refers to the link of two or more elements or components together through any means including chemical conjugation or recombination. In the case of polypeptides, two polymers of amino acid residues are directly coupled to each other, or in the case of polynucleotides, two polymers of nucleotides are directly coupled to each other, or separated by inserting amino acid residues or nucleotides within the same polypeptide or polynucleotide.

In the present application, the term “heterologous” generally refers to a polypeptide or nucleic acid containing two or more sequences or subsequences that have not been found in the same relationship with each other in nature. For example, recombination typically produces expression boxes that have two or more sequences from unrelated genes to arrange into new functional nucleic acids. The term “heterologous signal peptide” generally refers to the fact that in natural circumstances, the signal peptide and an amino acid sequence linked to the signal peptide do not belong to the same origin, may be from different species and different individuals, may be from the same species but different individuals, may be from the same individual but different proteins, and may be from the same species but different proteins. For example, the heterologous signal peptide and the amino acid sequence linked to the heterologous signal peptide are encoded by naturally unrelated genes to arrange into new polypeptides. For example, when a signal peptide is linked to an NGF protein or an NGF protein variant linked to the signal peptide, it can be said that the signal peptide is heterologous when the signal peptide and the NGF protein or the NGF protein variant are encoded by naturally unrelated genes, that is, when the gene encoding the signal peptide is not related to the gene encoding the NGF protein or the NGF protein variant, or when the protein linked to the signal peptide is not an NGF protein or an NGF protein variant in natural circumstances.

In the present application, the term “signal peptide” generally refers to a short peptide chain that guides the transfer of newly synthesized proteins to a secretion pathway.

The general length of a signal peptide is 5-30 amino acids. A signal peptide may be located at an N terminal or C terminal of a protein pro-form, but in most cases, it exists as an N-terminal peptide on the protein pro-form. The function of a signal peptide may be to promote the translocation of expressed polypeptides connected to the endoplasmic reticulum. A signal peptide is usually excised in this process. A signal peptide with a protein precursor excised may be heterologous or homologous to organisms used to produce polypeptides. For example, the signal peptide of the present application may contain functional active fragments, truncated bodies, and/or mutants of wild type signal peptides. For example, the signal peptide of the present application may have the function of guiding substances containing signal peptides to bind to cell membranes.

In the present application, the term “wild type” generally refers to a sequence of amino acids or nucleic acids that naturally exist within a species or population (such as human, mice, rats, and cells).

In the present application, the term “neurotrophin receptor p75” generally refers to a low-affinity nerve growth factor receptor (LNGFR), may bind to a neurotrophin and is a member of a tumor necrosis factor receptor superfamily, also known as a p75NTR. An amino acid sequence and other information of an exemplary human p75NTR can be found in the UniProtKB database login number P08138. As used herein, the p75NTR is not limited to a human p75NTR, and contains all species orthologs of the human p75NTR. The term “p75NTR” covers a pro-form of the p75NTR, a p75NTR precursor, a full-length p75NTR and any form of p75NTR produced in an intracellular process. The term also covers p75NTR variants that are present naturally, such as a splicing variant, an allelic variant and an isotype.

In the present application, the term “neurotrophin” generally refers to a protein that provides supports for survival, development and normal function realization of neurons. It is one of growth factors. An exemplary neurotrophin may include a nerve growth factor (NGF), a brain-derived neurotrophic factor (BDNF), a neurotrophin-3 (NT-3), a neurotrophin-4 (NT-4), a neurotrophin-6 (NT-6), a neurotrophin-7 (NT-7), dehydroepiandrosterone (DHEA), DHEA sulphate (DHEA-S), a GDNF ligand family, a ciliary neurotrophic factor (CNTF) and other biological molecules.

In the present application, the term “brain-derived neurotrophic factor (BDNF)” generally refers to one of neurotrophic factors, and may also be called “abrineurin”. The BDNF is a member of a neurotrophic factor growth factor family, and is related to a classic nerve growth factor. The BDNF is typically present in the brain and the periphery. As used herein, the BDNF is not limited to a human BDNF, and contains all species orthologs of the human BDNF. The term “BDNF” covers a pro-form of the BDNF, a BDNF precursor, a full-length BDNF and any form of BDNF produced in an intracellular process. The term also covers BDNF variants that are present naturally, such as a splicing variant, an allelic variant and an isotype. An amino acid sequence of a human BDNF protein can be found in the UniProtKB database login number P23560. A full-length human BDNF protein typically has 247 amino acids, and may contain a signal peptide, a precursor part, a leader peptide and a mature BDNF. The BDNF signal peptide may be amino acids at positions 1-18 of the full-length human BDNF protein, for example, the BDNF signal peptide may contain an amino acid sequence shown in SEQ ID NO: 31.

In the present application, the term “neurotrophin-3 (NT-3)” generally refers to a neurotrophic factor in a neurotrophic factor NGF (nerve growth factor) family. The NT-3 is a protein growth factor, and has activities on certain neurons of peripheral and central nervous systems. An amino acid sequence of an exemplary human NT-3 can be found in the UniProtKB database login number P20783.

In the present application, the term “neurotrophin-4 (NT-4)” generally refers to a neurotrophic factor in a neurotrophic factor NGF (nerve growth factor) family. The NT-4 sends a signal mainly through a TrkB receptor tyrosine kinase. The NT-4 may also be called an NT-5. An amino acid sequence of an exemplary human NT-4 can be found in the UniProtKB database login number P34130.

In the present application, the term “leader peptide” generally refers to an excised part in the mature or activation process of proteins. Once excised, the leader peptide usually does not have independent biological functions. The leader peptide may assist in folding and correct expression of proteins. In some cases, the leader peptide may contain a signal peptide or a part of the signal peptide. In some cases, the leader peptide does not contain a signal peptide.

In the present application, the term “delivery vector” generally refers to a transfer medium which can deliver a reagent (such as an mRNA) to a target cell. The delivery vector may deliver the reagent (such as the mRNA) to a specific cell subclass. For example, in virtue of the inherent features of the delivery vector or through a part coupled to the vector and a part contained in the delivery vector (or a part combined with the vector to maintain the part together with the delivery vector, thereby making the part sufficient to target the delivery vector), the delivery vector is made to target certain types of cells. The delivery vector may also increase the in-vivo half-life period of the reagent to be delivered (such as the mRNA) and/or the bioavailability of the reagent to be delivered. The delivery vector may include a viral vector, viral like particles, a polycationic vector, a peptide vector, liposome, and/or a hybrid vector. For example, if a target cell is a liver cell, the property of the delivery vector (such as the size, charges, and/or pH) may effectively deliver the delivery vector and/or molecules (such as the mRNA) encapsulated therein to the target cell, reduce immune clearance, and/or promote residence in the target cell.

In the present application, the term “lipid nanoparticle (LNP)” generally refers to a particle containing a plurality of (i.e., more than one) lipid molecules that physically bind to each other (such as covalently or non-covalently) through intermolecular force. The lipid nanoparticle may be, for example, a microsphere (including unilamellar and multilamellar vesicles, such as liposome), a dispersion phase in an emulsion, a micelle or an internal phase in a suspension. The lipid nanoparticle may contain one or more lipids (such as cationic lipids, non-cationic lipids and PEG-modified lipids).

In the present application, the term “liposome” generally refers to a vesicle having an internal space and isolated from an external medium through one or more double-layer membranes. For example, the double-layer membrane may be formed from amphiphilic molecules, such as synthesized or natural lipids containing spatially-isolated hydrophilic and hydrophobic domains; and for another example, the double-layer membrane may be formed from an amphiphilic polymer and a surfactant.

In the present application, when used for a nucleic acid (such as an RNA or a DNA), the term “modification” generally refers to, compared to a corresponding wild type, the nucleic acid has different nucleotide molecules, has different nucleotide sequences, is composed of different bonds and/or is doped with non-natural parts in its structure. For example, the modification may include modification to nucleotide, for example, the nucleotide may include a modified base, saccharide or phosphate group. For example, the modification may include polypeptides or proteins with different nucleotide sequences but encoding the same amino acid sequence, or polypeptides or proteins with the same function. The modification may be chemical modification and/or biological modification. “Chemical modification” may include the introduction of chemical substances that are different from those seen in wild type or naturally present nucleic acids, such as covalent modification, such as the introduction of modified nucleotides (such as nucleotide analogues, or the introduction of side groups that are not naturally found in these nucleic acid molecules). The term “modified nucleotide” usually refers to a unit in a nucleic acid polymer, and it contains a modified base, saccharide, or phosphate group, or is doped with non-natural parts in its structure.

In the present application, when used for a nucleic acid, the term “codon-optimization” generally represents a polypeptide-encoding nucleic acid that has been improved to have improved expression in a cell by replacing one, at least one, or one or more codons in a parental polypeptide-encoding nucleic acid with codons which have different relative use frequencies in a cell and encode the same amino acid residue.

In the present application, the term “polynucleotide” generally includes a DNA molecule (such as a cDNA or a genomic DNA), an RNA molecule (such as an mRNA), a DNA or RNA anologue produced using nucleotide analogues (such as a peptide nucleic acid and a non-naturally present nucleotide analogue), and their heterozygote. A nucleic acid molecule may be single-stranded or double-stranded.

In the present application, the term “isolated nucleic acid molecule” generally refers to a single or double stranded polymer or its analogues of deoxyribonucleotide or ribonucleotide bases read from the 5′ terminal to the 3′ terminal, and it has been isolated from at least about 50% of polypeptides, peptides, lipids, saccharides, polynucleotides, or other materials naturally discovered together with nucleic acid molecules when isolating total nucleic acids from source cells. For example, the isolated nucleic acid molecule generally does not contain any other polluting nucleic acid molecules or other molecules found in the natural environment of nucleic acids that can interfere with its use or its therapeutic, diagnostic, preventive, or research uses.

In the present application, the term “mRNA” generally refers to an RNA transcript that has been treated to remove introns and can be translated into polypeptides.

In the present application, the term “vector” generally refers to a nucleic acid molecule that can self-replicate in suitable hosts, and it transfers an inserted nucleic acid molecule to host cells and/or between the host cells. The vector may include a vector primarily used for inserting a DNA or an RNA into a cell, a vector primarily used for replicating a DNA or an RNA, and a vector primarily used for expression of transcription and/or translation of a DNA or an RNA. The vector also includes a vector with multiple aforementioned functions. The vector may be a polynucleotide that can be transcribed and translated into polypeptides when introduced into a suitable host cell. Usually, by culturing suitable host cells containing the vector, the vector may produce a desired expression product.

In the present application, the term “cell” generally refers to an individual cell, cell line, or cell culture that may or has already contained a plasmid or vector including the nucleic acid molecule described in the present application, or that can express antibodies or their antigen-binding fragments described in the present application. The cell may include a filial generation of a single host cell. Due to natural, accidental, or intentional mutations, a daughter cell may not necessarily be identical in morphology or genome to original parent cells, but can express the antibodies or their antigen-binding fragments described in the present application. The cell may be obtained by in vitro transfection of cells using the vector described in the present application. The cell may be a prokaryotic cell or a eukaryotic cell.

In the present application, the term “pharmaceutically acceptable carrier” generally includes a pharmaceutically acceptable carrier, excipient, or stabilizer that is non-toxic to cells or mammals exposed to it at the dosage and concentration adopted. Usually, a physiologically acceptable carrier is a pH buffered aqueous solution. Examples of the physiologically acceptable carrier may include a buffering agent, an antioxidant, a low molecular weight (less than about 10 residues) polypeptide, a protein, a hydrophilic polymer, an amino acid, monosaccharides, disaccharides, and other carbohydrates, a chelating agent, sugar alcohols, and salt forming counter ions, such as sodium; and/or a non-ionic surfactant.

In the present application, the term “pharmaceutical composition” generally refers to a preparation in the form allowing the biological activity of active ingredients (such as an S-protein variant and the nucleic acid molecule of the present application) to be effective, and it does not contain other ingredients that have unacceptable toxicity to a subject to which the preparation is to be administrated. The preparation may be sterile.

In the present application, the term “kit” generally refers to a packaged product containing components used to treat PD-1 mediated related diseases by administrating antigen binding proteins of the present application. The components of the kit may be contained in separate vials (i.e., a kit with separate parts) or provided in a single vial. The kit may include reagents such as buffering agents, protein stabilizing reagents, signal generation systems (such as fluorescence signal generation systems), antibodies, control proteins, and test containers. The kit may also contain instructions for implementing the method.

In the present application, the term “drug administration device” includes: (i) an infusion module for administering a pharmaceutical composition containing an active ingredient to a subject; (ii) a pharmaceutical composition for infusion, the pharmaceutical composition containing an active ingredient selected from: a recombinant protein, a nucleic acid molecule, an mRNA, a vector, a cell, a composition, a pharmaceutical composition, or a combinations thereof; and (iii) an optional drug efficacy monitoring module.

In the present application, the term “central and/or peripheral nervous system disease” generally refers to diseases related to lesions of a central nervous system (brain or spinal cord) and a peripheral nervous system (nerves outside the brain or spinal cord). For example, the central and/or peripheral nervous system disease may manifest as being caused by one or more of the following: neuronal degeneration, inflammation or loss of oligodendrocytes, cerebral blood supply infarction, injury or tumor and bacterial or viral infections. The central and/or peripheral nervous system disease may include Alzheimer's disease, Huntington's chorea, Parkinson's disease, multiple sclerosis, encephalitis or meningitis, damage to brain or spinal cord structures, and/or stroke.

In the present application, the term “neurodegenerative disease” generally refers to the gradual loss of neuronal functions and structures leading to cognitive impairment. The neurodegenerative disease may include Alzheimer's disease, Parkinson's disease, Huntington's chorea, early-onset Alzheimer's disease or early-onset Parkinson's disease, and/or amyotrophic lateral sclerosis.

In the present application, the term “peripheral neuropathy” generally refers to a disease or condition caused by abnormalities in a peripheral nervous system. The peripheral nervous system includes all nerves except the central nervous system (brain and spinal cord), such as nerves (brain nerves) that connect the head, face, eyes, nose, muscles, and ears to the brain, nerves that connect the spinal cord to other parts of the body, and nerve cells distributed throughout various parts of the body.

In the present application, the term “subject” generally refers to human or non-human animals (including mammals) that require diagnosis, prognosis, improvement, prevention, and/or treatment of diseases, such as human, non-human primates (apes, gibbons, gorillas, chimpanzees, orangutans, and macaques), domestic animals (dogs and cats), farm animals (poultry such as chickens and ducks, horses, cows, goats, sheep, and pigs), and experimental animals (mice, rats, rabbits, and guinea pigs). Human subjects include fetuses, newborns, infants, adolescents, and adult subjects. The subject may include an animal disease model.

In the present application, the terms “include”, “comprise”, “have”, “may”, “contain” and their variants are typically intended to be open-ended transitional phrases, terms, or words, and do not exclude the possibility of additional behaviors or structures. The term “composed of” generally represents that there cannot be other components (or similarly, features, integers, steps, etc.). Unless otherwise explicitly stated in the context, singular forms such as “a”, “an”, and “the” in English, and “a”, “an”, and “said/the” in Chinese generally include the plural form of the thing referred to. In the present application, the term “about” generally refers to approximately, in the region of, roughly, or around. When the term “about” is used to refer to a range of numerical values, a cutoff or specific value is used to indicate that the stated value may differ by up to 10% from the listed values. Therefore, the term “about” may be used to cover variations of 10% or less, ±5% or less, ±1% or less, ±0.5% or less, or ±0.1% or less from a specific value.

### DETAILED DESCRIPTION OF THE PRESENT INVENTION

### Recombinant Protein

In one aspect, the present application provides a recombinant protein, and the recombinant protein includes a nerve growth factor (NGF) protein variant, wherein compared with a wild type NGF protein, binding of the NGF protein variant to a neurotrophin receptor p75 (p75NTR) is reduced by at least about 50% (for example, reduced by at least about 55%, at least about 60%, at least about 65%, at least about 70%, at least about 75%, at least about 80%, at least about 85%, at least about 90%, at least about 95%, at least about 98% or above), for example, the wild type NGF protein may contain an amino acid sequence shown in SEQ ID NO: 32.

In the present application, compared with the amino acid sequence shown in SEQ ID NO: 32, the NGF protein variant may contain one or more amino acid mutations. For example, compared with the amino acid sequence shown in SEQ ID NO: 32, the NGF protein variant may contain an amino acid mutation at R100 (i.e., arginine at position 100). For example, compared with the amino acid sequence shown in SEQ ID NO: 32, arginine at position 100 of the NGF protein variant is mutated to tryptophan (i.e., R100W). For example, the NGF protein variant contains an amino acid sequence shown in SEQ ID NO: 33.

It has been known in the art that when a mature NGF protein has R100W mutation, its ability of being secreted out of a cell is lowered. The present application provides a recombinant protein of an NGF^{R100W} variant with an expression amount and/or secretion amount increased compared with a naturally present NGF^{R100W} variant protein. The recombinant protein of the present application can increase the amount of secretion out of a cell of a mature NGF^{R100W} variant protein. For example, the recombinant protein of the present application may contain a heterologous signal peptide, and the heterologous signal peptide may be located at the N terminal of the NGF protein variant. For example, compared with a signal peptide shown in SEQ ID NO: 29, the heterologous signal peptide increases the expression amount and/or the secretion amount of the NGF protein variant by at least about 50% (for example, increased by at least about 55%, at least about 60%, at least about 65%, at least about 70%, at least about 75%, at least about 80%, at least about 85%, at least about 90%, at least about 95%, at least about 98% or above).

The heterologous signal peptide of the present application may have a length of 5-30 amino acids. For example, the heterologous signal peptide of the present application may have a length of 5-25 amino acids. For example, the heterologous signal peptide of the present application may have a length of 5-20 amino acids. For example, the heterologous signal peptide of the present application may have a length of 10-25 amino acids. For example, the heterologous signal peptide of the present application may have a length of 10-30 amino acids. For example, the heterologous signal peptide of the present application may have a length of 15-30 amino acids. For example, the heterologous signal peptide of the present application may have a length of 10-20 amino acids. For example, the heterologous signal peptide of the present application may have a length of 15-20 amino acids. For example, the heterologous signal peptide of the present application may have a length of 18 amino acids. For example, the heterologous signal peptide of the present application may have a length of 19 amino acids. For example, the heterologous signal peptide of the present application may have a length of 20 amino acids.

The heterologous signal peptide of the present application may come from prokaryotes, such as bacteria (E. coli, Staphylococcus aureus, and Bacillus subtilis). The heterologous signal peptide of the present application may come from eukaryotes, such as yeast or mammalian cells (human, mice, monkeys, etc.).

The heterologous signal peptide of the present application may be derived from secretory proteins, periplasmic proteins, and/or membrane binding proteins, such as enzymes. For example, the signal peptide may be derived from E. coli outer membrane proteins A OmpA, OmpF, a X bacteriophage receptor LamB, heat stable enterotoxin ST, alkaline phosphatase PhoA, maltose binding proteins MBP, DsbA, Staphylococcus aureus proteins A, Bacillus subtilis protease, neutral protease, Bacillus marginalis RNA enzymes, and levanase. For example, the signal peptide may also be derived from natural signal peptides of exogenous proteins in yeast expression systems, α-factor signal peptides, sucrase gene SUC signal peptide sequences, acid phosphatase genes PHO1 and/or interstitial metalloproteinase, or derived from human serum proteins and bovine chymosin.

The heterologous signal peptide of the present application may be derived from Gaussia, Homo sapiens, Oikopleura dioica or Mus musculus. The heterologous signal peptide of the present application may contain a signal peptide derived from Gaussia luciferase, human trypsinogen-2, Oikosin 1 mutants, azurocidin preproprotein, and/or Igx. In certain implementations, the heterologous signal peptide is derived from Gp67, Gp64, honeybee melittin (HBM), albumin, IL-2, azurocidin preproprotein, mouse IgK, an immunoglobulin heavy chain and/or a cystatin-S precursor.

The heterologous signal peptide of the present application may contain a signal peptide derived from neurotrophins. For example, the heterologous signal peptide may be a signal peptide of a brain-derived neurotrophic factor (BDNF), a neurotrophin-3 (NT-3) and/or a neurotrophin-4 (NT-4). For example, the heterologous signal peptide may contain an amino acid sequence shown in any one of SEQ ID NOs: 30, 31 and 35-43. For example, the heterologous signal peptide is an IgK signal peptide and/or a BDNF signal peptide. For example, the heterologous signal peptide contains an amino acid sequence shown in any one of SEQ ID NOs: 30-31.

For example, the heterologous signal peptide is an IgK signal peptide, and for another example, the heterologous signal peptide contains an amino acid sequence shown in SEQ ID NO: 30.

For example, the heterologous signal peptide is a BDNF signal peptide, and for another example, the heterologous signal peptide contains an amino acid sequence shown in SEQ ID NO: 31.

For example, the heterologous signal peptide is an hIL-2 signal peptide, and for another example, the heterologous signal peptide contains an amino acid sequence shown in SEQ ID NO: 35.

For example, the heterologous signal peptide is an APP signal peptide, and for another example, the heterologous signal peptide contains an amino acid sequence shown in SEQ ID NO: 36.

For example, the heterologous signal peptide is an HC signal peptide, and for another example, the heterologous signal peptide contains an amino acid sequence shown in SEQ ID NO: 37.

For example, the heterologous signal peptide is a Cystatin signal peptide, and for another example, the heterologous signal peptide contains an amino acid sequence shown in SEQ ID NO: 38.

For example, the heterologous signal peptide is a Gp67 signal peptide, and for another example, the heterologous signal peptide contains an amino acid sequence shown in SEQ ID NO: 39.

For example, the heterologous signal peptide is an HBM signal peptide, and for another example, the heterologous signal peptide contains an amino acid sequence shown in SEQ ID NO: 40.

For example, the heterologous signal peptide is a Gp64 signal peptide, and for another example, the heterologous signal peptide contains an amino acid sequence shown in SEQ ID NO: 41.

For example, the heterologous signal peptide is an Alb signal peptide, and for another example, the heterologous signal peptide contains an amino acid sequence shown in SEQ ID NO: 42.

For example, the heterologous signal peptide is an SP signal peptide, and for another example, the heterologous signal peptide contains an amino acid sequence shown in SEQ ID NO: 43.

In the present application, the recombinant protein further includes a leader peptide. The leader peptide may be a leader peptide of an NGF protein, or a heterologous leader peptide derived from a non-NGF protein. For example, the leader peptide may contain an amino acid sequence shown in SEQ ID NO: 34.

In the present application, the recombinant protein further includes a leader peptide. In the present application, from an N terminal to a C terminal, the recombinant protein may sequentially include the heterologous signal peptide, the leader peptide and the NGF protein variant.

In the present application, the recombinant protein further includes a leader peptide. In the present application, from an N terminal to a C terminal, the recombinant protein may sequentially include the heterologous signal peptide, the leader peptide and the NGF protein variant, the heterologous signal peptide contains an amino acid sequence shown in any one of SEQ ID NOs: 30-31 and 35-43, the leader peptide contains an amino acid sequence shown in SEQ ID NO: 34, and the NGF protein variant contains an amino acid sequence shown in SEQ ID NO: 33.

For example, from the N terminal to the C terminal, the recombinant protein may sequentially include the IgK signal peptide, the leader peptide and the NGF protein variant. For example, the recombinant protein contains an amino acid sequence shown in SEQ ID NO: 15.

For example, from the N terminal to the C terminal, the recombinant protein may sequentially include the IgK signal peptide and the NGF protein variant. For example, the recombinant protein contains an amino acid sequence shown in SEQ ID NO: 16.

For example, from the N terminal to the C terminal, the recombinant protein may sequentially include the BDNF signal peptide, the leader peptide and the NGF protein variant. For example, the recombinant protein contains an amino acid sequence shown in SEQ ID NO: 19.

For example, from the N terminal to the C terminal, the recombinant protein may sequentially include the BDNF signal peptide and the NGF protein variant. For example, the recombinant protein contains an amino acid sequence shown in SEQ ID NO: 20.

For example, from the N terminal to the C terminal, the recombinant protein may sequentially include the SP signal peptide, the leader peptide and the NGF protein variant. For example, the recombinant protein contains an amino acid sequence shown in SEQ ID NO: 58.

For example, from the N terminal to the C terminal, the recombinant protein may sequentially include the hIL-2 signal peptide, the leader peptide and the NGF protein variant. For example, the recombinant protein contains an amino acid sequence shown in SEQ ID NO: 59.

For example, from the N terminal to the C terminal, the recombinant protein may sequentially include the Cystatin signal peptide, the leader peptide and the NGF protein variant. For example, the recombinant protein contains an amino acid sequence shown in SEQ ID NO: 60.

For example, from the N terminal to the C terminal, the recombinant protein may sequentially include the APP signal peptide, the leader peptide and the NGF protein variant. For example, the recombinant protein contains an amino acid sequence shown in SEQ ID NO: 61.

For example, from the N terminal to the C terminal, the recombinant protein may sequentially include the HC signal peptide, the leader peptide and the NGF protein variant. For example, the recombinant protein contains an amino acid sequence shown in SEQ ID NO: 62.

For example, from the N terminal to the C terminal, the recombinant protein may sequentially include the Gp67 signal peptide, the leader peptide and the NGF protein variant. For example, the recombinant protein contains an amino acid sequence shown in SEQ ID NO: 63.

For example, from the N terminal to the C terminal, the recombinant protein may sequentially include the HBM signal peptide, the leader peptide and the NGF protein variant. For example, the recombinant protein contains an amino acid sequence shown in SEQ ID NO: 64.

For example, from the N terminal to the C terminal, the recombinant protein may sequentially include the Gp64 signal peptide, the leader peptide and the NGF protein variant. For example, the recombinant protein contains an amino acid sequence shown in SEQ ID NO: 65.

For example, from the N terminal to the C terminal, the recombinant protein may sequentially include the Alb signal peptide, the leader peptide and the NGF protein variant. For example, the recombinant protein contains an amino acid sequence shown in SEQ ID NO: 66.

In certain implementations, the recombinant protein further includes a tag peptide.

For example, from the N terminal to the C terminal, the recombinant protein may sequentially include the heterologous signal peptide, the leader peptide, the NGF protein variant and the tag peptide.

For example, from the N terminal to the C terminal, the recombinant protein may sequentially include the heterologous signal peptide, the NGF protein variant and the tag peptide.

For example, the tag peptide may contain an amino acid sequence shown in (YPYDVPDYA)n, wherein n is any integer from 1 to 5. For another example, the tag peptide may contain an amino acid sequence shown in YPYDVPDYA (SEQ ID NO: 68) or YPYDVPDYAYPYDVPDYA (SEQ ID NO: 69).

For example, from the N terminal to the C terminal, the recombinant protein may sequentially include the IgK signal peptide, the leader peptide, the NGF protein variant and the tag peptide. For example, the recombinant protein contains an amino acid sequence shown in SEQ ID NO: 70.

In certain implementations, the recombinant protein further includes a transmembrane anchoring region.

For example, from the N terminal to the C terminal, the recombinant protein may sequentially include the heterologous signal peptide, the leader peptide, the NGF protein variant and the transmembrane anchoring region.

For example, from the N terminal to the C terminal, the recombinant protein may sequentially include the heterologous signal peptide, the NGF protein variant and the transmembrane anchoring region.

For example, the transmembrane anchoring region may be a C-terminal transmembrane anchoring domain of a platelet-derived growth factor receptor (PDGFR). For another example, the transmembrane anchoring region may contain an amino acid sequence shown in SEQ ID NO: 67.

For example, from the N terminal to the C terminal, the recombinant protein may sequentially include the IgK signal peptide, the leader peptide, the NGF protein variant and the transmembrane anchoring region. For example, the recombinant protein contains an amino acid sequence shown in SEQ ID NO: 17.

For example, from the N terminal to the C terminal, the recombinant protein may sequentially include the IgK signal peptide, the NGF protein variant and the transmembrane anchoring region. For example, the recombinant protein contains an amino acid sequence shown in SEQ ID NO: 18.

In certain implementations, the recombinant protein further includes a domain prolonging a half-life period.

For example, the recombinant protein further includes an Fc region of immunoglobulin. The Fc region may prolong the half-life period of the recombinant protein. The Fc region may be located at the C terminal of the recombinant protein.

For example, compared with a recombinant protein which does not contain an Fc region, the half-life period of the recombinant protein containing the Fc region may be prolonged by at least about 10%, 20%, 30%, 40%, 50%, 60%, 70%, 80%, 90% or above.

For example, the Fc region may be derived from human immunoglobulin and/or mouse immunoglobulin.

For example, the Fc region may be an Fc region of human IgG1.

For example, the Fc region may be an Fc region of mouse IgG.

For example, the Fc region may contain an amino acid sequence shown in any one of SEQ ID NOs: 56-57.

For example, from the N terminal to the C terminal, the recombinant protein may sequentially include the heterologous signal peptide, the leader peptide, the NGF protein variant and the Fc region.

For another example, from the N terminal to the C terminal, the recombinant protein may sequentially include the IgK signal peptide, the leader peptide, the NGF protein variant and the Fc region.

For example, the recombinant protein may contain an amino acid sequence shown in any one of SEQ ID NOs: 54-55.

### Nucleic Acid Molecule

In another aspect, the present application provides an isolated nucleic acid molecule, and the isolated nucleic acid molecule encodes the recombinant protein. For example, the isolated nucleic acid molecule may encode a recombinant protein containing an amino acid sequence shown in any one of SEQ ID NOs: 15-20, 54-55 and 58-66.

For example, the isolated nucleic acid molecule may encode a recombinant protein containing an amino acid sequence shown in SEQ ID NO: 15. For example, the isolated nucleic acid molecule may encode a recombinant protein containing an amino acid sequence shown in SEQ ID NO: 19.

For example, the isolated nucleic acid molecule may include a DNA and/or an RNA. For example, the isolated nucleic acid molecule may be a DNA, and may encode a recombinant protein containing an amino acid sequence shown in SEQ ID NO: 15 or 19. For example, the isolated nucleic acid molecule may be an RNA, and may encode a recombinant protein containing an amino acid sequence shown in SEQ ID NO: 15 or 19.

For example, the isolated nucleic acid molecule may contain modifications at one or more positions selected from: a 5′ cap, a 5′ untranslated region, an open reading frame, a 3′ untranslated region, and a poly A tail.

For example, the isolated nucleic acid molecule may contain at least one modified nucleotide.

In the present application, the nucleic acid molecule may contain a nucleotide sequence shown in any one of SEQ ID NOs: 2-8, 10-11, 23-28, 46-47 and 50-51. In the present application, the nucleic acid molecule may contain a nucleotide sequence having the sequence identity of at least 80% (such as 82%, 85%, 88%, 90%, 95%, 98%, 99% or above) with the nucleotide sequence shown in any one of SEQ ID NOs: 2-8, 10-11, 23-28, 46-47 and 50-51.

In the present application, the nucleic acid molecule may contain a nucleotide sequence shown in any one of SEQ ID NOs: 11-12. In the present application, the nucleic acid molecule may contain a nucleotide sequence having the sequence identity of at least 80% (such as 82%, 85%, 88%, 90%, 95%, 98%, 99% or above) with the nucleotide sequence shown in any one of SEQ ID NOs: 11-12.

In the present application, the nucleic acid molecule may contain a nucleotide sequence shown in any one of SEQ ID NOs: 3, 11 and 23. In the present application, the nucleic acid molecule may contain a nucleotide sequence having the sequence identity of at least 80% (such as 82%, 85%, 88%, 90%, 95%, 98%, 99% or above) with the nucleotide sequence shown in any one of SEQ ID NOs: 3, 11 and 23.

In the present application, the nucleic acid molecule may contain a nucleotide sequence shown in any one of SEQ ID NOs: 7, 12 and 27. In the present application, the mRNA may contain a nucleotide sequence having the sequence identity of at least 80% (such as 82%, 85%, 88%, 90%, 95%, 98%, 99% or above) with the nucleotide sequence shown in any one of SEQ ID NOs: 7, 12 and 27.

In one aspect, the present application provides an isolated RNA, particularly an mRNA. The mRNA contains a nucleic acid molecule which can encode a nerve growth factor (NGF) protein or a variant thereof, or, at least a part of the mRNA can encode the NGF protein variant.

In certain implementations, the NGF protein contains an amino acid sequence shown in SEQ ID NO: 32.

In certain implementations, compared with the amino acid sequence shown in SEQ ID NO: 32, binding of the NGF protein variant to a neurotrophin receptor p75 (p75NTR) is reduced by at least 50% (for example, reduced by at least 55%, at least 60%, at least 65%, at least 70%, at least 75%, at least 80%, at least 85%, at least 90%, at least 95%, at least 98% or above).

In certain implementations, the NGF protein variant contains an amino acid sequence shown in SEQ ID NO: 33.

In certain implementations, the mRNA contains a polynucleotide encoding a heterologous signal peptide, and compared with a signal peptide shown in SEQ ID NO: 29, the heterologous signal peptide increases an expression amount and/or a secretion amount of the NGF protein variant by at least 50% (for example, increased by at least 55%, at least 60%, at least 65%, at least 70%, at least 75%, at least 80%, at least 85%, at least 90%, at least 95%, at least 98% or above).

In certain implementations, the heterologous signal peptide enables the expression amount and/or the secretion amount of the NGF protein variant to reach 50% or above (for example, reach at least 55%, at least 60%, at least 65%, at least 70%, at least 75%, at least 80%, at least 85%, at least 90%, at least 95%, at least 98% or above) of those of a wild type NGF protein.

In certain implementations, the heterologous signal peptide is derived from a neurotrophin.

In certain implementations, the heterologous signal peptide is derived from a brain-derived neurotrophic factor (BDNF), a neurotrophin-3 (NT-3) and/or a neurotrophin-4 (NT-4).

In certain implementations, the heterologous signal peptide is derived from Gp67, Gp64, honeybee melittin (HBM), albumin, IL-2, azurocidin preproprotein, mouse IgK, an immunoglobulin heavy chain and/or a cystatin-S precursor.

In certain implementations, the heterologous signal peptide is an IgK signal peptide and/or a BDNF signal peptide.

In certain implementations, the heterologous signal peptide contains an amino acid sequence shown in any one of SEQ ID NOs: 30-31 and 35-43.

In certain implementations, the heterologous signal peptide contains an amino acid sequence shown in any one of SEQ ID NOs: 30-31.

In certain implementations, the mRNA further includes a polynucleotide encoding a leader peptide.

In certain implementations, the leader peptide contains an amino acid sequence shown in SEQ ID NO: 34.

In certain implementations, from a 5′ terminal to a 3′ terminal, the mRNA sequentially includes the polynucleotide encoding the heterologous signal peptide, the polynucleotide encoding the leader peptide, and a polynucleotide encoding the NGF protein or the variant thereof.

In certain implementations, from the 5′ terminal to the 3′ terminal, the mRNA sequentially includes a polynucleotide encoding the IgK signal peptide, the polynucleotide encoding the leader peptide, and the polynucleotide encoding the NGF protein variant.

In certain implementations, from the 5′ terminal to the 3′ terminal, the mRNA sequentially includes a polynucleotide encoding the BDNF signal peptide, the polynucleotide encoding the leader peptide, and the polynucleotide encoding the NGF protein variant.

In certain implementations, the mRNA encodes and contains an amino acid sequence shown in any one of SEQ ID NOs: 15-20. In certain implementations, the mRNA encodes and contains an amino acid sequence shown in any one of SEQ ID NOs: 15 and 19.

In certain implementations, the recombinant protein further includes a transmembrane anchoring region.

For example, from the N terminal to the C terminal, the recombinant protein may sequentially include the heterologous signal peptide, the leader peptide, the NGF protein variant and the transmembrane anchoring region.

For example, from the N terminal to the C terminal, the recombinant protein may sequentially include the heterologous signal peptide, the NGF protein variant and the transmembrane anchoring region.

For example, the transmembrane anchoring region may be a PDGFR transmembrane anchoring region. For another example, the transmembrane anchoring region may contain an amino acid sequence shown in SEQ ID NO: 67.

For example, from the N terminal to the C terminal, the recombinant protein may sequentially include the IgK signal peptide, the leader peptide, the NGF protein variant and the transmembrane anchoring region. For example, from the N terminal to the C terminal, the recombinant protein may sequentially include the IgK signal peptide, the NGF protein variant and the transmembrane anchoring region.

In certain implementations, the mRNA encodes and contains an amino acid sequence shown in any one of SEQ ID NOs: 17-18.

In certain implementations, the mRNA further contains a polynucleotide encoding an Fc region of immunoglobulin.

In certain implementations, the Fc region is derived from human immunoglobulin and/or mouse immunoglobulin.

In certain implementations, the Fc region is an Fc region of human IgG1.

In certain implementations, the Fc region is an Fc region of mouse IgG.

In certain implementations, the Fc region contains an amino acid sequence shown in any one of SEQ ID NOs: 56-57.

In certain implementations, from the 5′ terminal to the 3′ terminal, the mRNA sequentially includes the polynucleotide encoding the heterologous signal peptide, the polynucleotide encoding the leader peptide, the polynucleotide encoding the NGF protein variant, and the polynucleotide encoding the Fc region of the immunoglobulin.

In certain implementations, from the 5′ terminal to the 3′ terminal, the mRNA sequentially includes the polynucleotide encoding the IgK signal peptide, the polynucleotide encoding the leader peptide, the polynucleotide encoding the NGF protein variant, and the polynucleotide encoding the Fc region of the immunoglobulin.

In certain implementations, the mRNA encodes and contains an amino acid sequence shown in any one of SEQ ID NOs: 54-55.

In one aspect, the present application provides an isolated RNA, particularly an mRNA. The mRNA contains a nucleic acid molecule which can encode the recombinant protein, or, at least a part of the mRNA can encode the recombinant protein, for example, the recombinant protein may contain an amino acid sequence shown in SEQ ID NO: 15.

In the present application, the RNA may be a naturally or non-naturally present RNA, such as the mRNA. The mRNA may include one or more nucleic bases, nucleosides or nucleotides. In the present application, the term “nucleoside” generally refers to a compound containing sugar molecules (such as pentose or ribose) or derivatives and organic bases (such as purine and pyrimidine) thereof or a combination of derivatives (also called “nucleic bases” herein) thereof. In the present application, a “nucleotide” generally refers to a nucleoside including a phosphate group.

In the present application, the mRNA may include a 5′ untranslated region (5′ UTR), a 3′ untranslated region (3′ UTR) and/or a coding region (such as an open reading frame).

In the present application, the mRNA may be modified. The modification may be chemical modification or biological modification. In the present application, the mRNA may contain one or more modified nucleic bases, nucleosides or nucleotides, and at the moment, it may be called a “chemically modified mRNA”, and may also be called a “modified mRNA” herein. Compared with a non-modified reference sequence (such as a naturally present or wild type mRNA), the modified mRNA may have useful properties, e.g., including enhanced stability, improved intracellular retention, an enhanced translation effect and/or lowered immunogenicity. Therefore, using the modified mRNA may improve the protein production efficiency, improve the retention of nucleic acids in cells and lower immunogenicity.

In certain implementations, the mRNA contains modifications at one or more positions selected from: a 5′ cap, a 5′ untranslated region, an open reading frame, a 3′ untranslated region, and a poly A tail.

In the present application, the mRNA may include a modified nucleic base, which may be modified uracil. In the present application, the mRNA may include a modified nucleic base, which may be modified cytosine. In the present application, the mRNA may include a modified nucleic base, which may be modified adenine. In the present application, the mRNA may include a modified nucleic base, which may be modified guanine. In the present application, the mRNA may include a combination of one or more aforementioned modified nucleic bases (such as 2, 3 or 4 combinations of the aforementioned modified nucleic bases).

For example, the isolated nucleic acid molecule may be one or more nucleotides selected from: N1-methylpseudouridine-5′-triphosphate (N1-Methylpseudo-UTP), pseudouridine-5′-triphosphate (pseudo-UTP), 5-methoxyuridine-5′-triphosphate (5-Methoxy-UTP) and 5-methylcytidine-5′-triphosphate (5-Methyl-CTP). In certain embodiments, the modified RNA of the present application includes a combination of one or more aforementioned modified nucleic bases (such as 2, 3 or 4 combinations of the aforementioned modified nucleic bases).

In the present application, the mRNA may be codon-optimized, and a codon still encodes the same amino acid after one or more nucleotides of the codon are changed.

In the present application, the mRNA may contain a nucleotide sequence shown in any one of SEQ ID NOs: 23-28 and 50-51. In the present application, the mRNA may contain a nucleotide sequence having the sequence identity of at least 80% (such as 82%, 85%, 88%, 90%, 95%, 98%, 99% or above) with the nucleotide sequence shown in any one of SEQ ID NOs: 23-28 and 50-51.

In the present application, the mRNA may contain a nucleotide sequence shown in SEQ ID NO: 23. In the present application, the mRNA may contain a nucleotide sequence having the sequence identity of at least 80% (such as 82%, 85%, 88%, 90%, 95%, 98%, 99% or above) with the nucleotide sequence shown in SEQ ID NO: 23.

In the present application, the mRNA may contain a nucleotide sequence shown in SEQ ID NO: 23. In the present application, the mRNA may contain a nucleotide sequence having the sequence identity of at least 80% (such as 82%, 85%, 88%, 90%, 95%, 98%, 99% or above) with the nucleotide sequence shown in SEQ ID NO: 23.

In the present application, the mRNA may contain a nucleotide sequence shown in SEQ ID NO: 27. In the present application, the mRNA may contain a nucleotide sequence having the sequence identity of at least 80% (such as 82%, 85%, 88%, 90%, 95%, 98%, 99% or above) with the nucleotide sequence shown in SEQ ID NO: 27.

The mRNA of the present application may be produced through any feasible method in the art, including but not limited to in vitro transcription (IVT) and synthesis methods. The mRNA may be prepared using enzymatic, solid-phase, liquid-phase, combinatorial synthesis methods, small domain synthesis, and ligation methods. In one embodiment, the mRNA is prepared using an IVT enzymatic synthesis method. Therefore, the present application further relates to polynucleotides, such as DNAs, constructs, and vectors, that may also be used for in vitro transcription of the mRNA in the present application.

### Composition

In the present application, the composition may further contain a delivery vector. The mRNA of the present application may be prepared in nanoparticles or other delivery vectors, for example, to avoid being degraded when delivered to a subject. In the present application, the mRNA may be encapsulated in nanoparticles. In a specific embodiment, the nanoparticles are particles with at least one size (such as a diameter) being less than or equal to about 1000 nM, less than or equal to about 500 nM, less than or equal to about 400 nM, less than or equal to about 300 nM, less than or equal to about 200 nM, or less than or equal to about 100 nM. In a specific embodiment, the nanoparticles may include lipids. Lipid nanoparticles may include, but are not limited to liposome and micelles. In the present application, the lipid nanoparticles may include cationic and/or ionizable lipids, anionic lipids, non-cationic lipids, neutral lipids, amphiphilic lipids, polyethylene glycolated lipids, and/or structural lipids, or a combination of the above. In certain embodiments, the lipid nanoparticles contain one or more RNAs, such as the mRNA, as described in the present application, for another example, an mRNA encoding a target polypeptide (such as the recombinant protein or the NGF protein variant). For example, the lipid nanoparticles contain one or more mRNAs encoding the recombinant protein or the NGF protein variant of the present application.

The delivery vector in the composition of the present application may be the lipid nanoparticles. The lipid nanoparticles may contain one or more (such as 1, 2, 3, 4, 5, 6, 7 or 8) cationic and/or ionizable lipids. The “cationic lipid” generally refers to a lipid carrying any number of net positive charges under certain pH (such as physiological pH). The cationic lipid may include, but is not limited to 3-(didodecylamino)-N1,N1,4-dotriacontyl-1-piperazine ethylamine (KL10), N1-[2-(docosylamino)ethyl]-N1,N4,N4-dotriacontyl-1,4-piperazine diethylamine (KL22), 14,25-tricosyl-15,18,21,24-tetraazaoctaporealkane (KL25), DLin-DMA, DLin-K-DMA, DLin-KC2-DMA, Octyl-CLinDMA, Octyl-CLinDMA (2S), DODAC, DOTMA, DDAB, DOTAP, DOTAP.C1, DC-Choi, DOSPA, DOGS, DODAP, DODMA and DMRIE. In addition, many commercial cationic and/or ionizable lipids may be used, such as LIPOFECTIN® (including DOTMA and DOPE) and LIPOFECTAMINE® (including DOSPA and DOPE). For example, the cationic lipid may be DLin-MC3-DMA or DLin-KC2-DMA.

In certain implementations, a molar ratio of the cationic lipid in the lipid nanoparticles is about 40%-70%, such as about 40%-65%, about 40%-60%, about 45%-55% or about 48%-53%. In certain implementations, a molar ratio of the cationic lipid (such as DLin-MC3-DMA) in the lipid nanoparticles is about 50%.

In the present application, the lipid nanoparticles may contain one or more (such as 1, 2, 3, 4, 5, 6, 7 or 8) non-cationic lipids. The non-cationic lipids may include anionic lipids. The anionic lipids suitable for the lipid nanoparticles of the present application may include phosphatidylglycerol, cardiolipin, diacylphosphatidylserine, diacylphosphatidic acid, N-lauroylphosphatidylethanolamine, N-succinylphosphatidylethanolamine, N-glutarylphosphatidylphosphoethanol, and other neutral lipids to which anionic groups are linked.

The non-cationic lipids may include neutral lipids. The neutral lipids suitable for the lipid nanoparticles of the present application may include phospholipid, such as distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoylphosphatidylethanolamine (DOPE), palmitoyloleoylphosphatidylcholine (POPC), palmitoyloleoyl-Phosphatidylethanolamine (POPE), dioleoyl-phosphatidylethanolamine 4-(N-maleimidomethyl)-cyclohexane-1-carboxylate (DOPE-mal), dipalmitoylphosphatidylethanolamine (DPPE), dimyristoylphosphoethanolamine (DMPE), distearoyl-phosphatidyl-ethanolamine (DSPE), 16-O-monomethylPE, 16-O-dimethylPE, 18-1-transPE, 1-stearoyl-2-oleoyl-phosphatidylethanolamine (SOPE), or mixtures thereof. In addition, lipids having a mixture of saturated and unsaturated fatty acid chains may be used. For example, the neutral lipids in the present application may be selected from DOPE, DSPC, DPPC, POPC or any related phosphatidylcholine.

In certain implementations, a molar ratio of the phospholipid in the lipid nanoparticles is about 30%-45%, such as about 33%-42%, about 35%-40%, or about 38%-39%. In certain implementations, a molar ratio of the phospholipid (such as DSPC) in the lipid nanoparticles is about 38.5%.

In the present application, the lipid nanoparticles may contain a lipid conjugate, such as a polyethylene glycol (PEG) modified lipid and a derived lipid. The PEG-modified lipid may include, but is not limited to a polyethylene glycol chain up to 5 kDa in length covalently linked to a lipid having an alkyl chain of a C6-C20 length. The addition of these components may prevent lipid aggregation, increase circulation duration, facilitate delivery of lipid-nucleic acid compositions to target cells, or rapidly release nucleic acids. For example, the polyethylene glycol (PEG) modified lipid molecule may be a PEG-ceramide with a shorter acyl chain (e.g., C14 or C18). For example, the lipid nanoparticles may contain PEG2000-DMG.

In certain implementations, a molar ratio of the polyethylene glycol (PEG) modified lipid molecule in the lipid nanoparticles is about 0.5%-2%, such as about 1%-2%, about 1.2%-1.8%, or about 1.4%-1.6%. In certain implementations, a molar ratio of the polyethylene glycol (PEG) modified lipid molecule (such as PEG2000-DMG) in the lipid nanoparticles is about 1.5%.

In the present application, the lipid nanoparticles may also contain cholesterol. In certain implementations, a molar ratio of the cholesterol in the lipid nanoparticles is about 5%-15%, such as about 6%-14%, about 7%-13%, about 8%-12% or about 9%-11%. In certain implementations, a molar ratio of the cholesterol in the lipid nanoparticles is about 10%.

In the present application, the lipid nanoparticles may include the cationic lipids, the cholesterol, the phospholipid, and the polyethylene glycol modified lipid molecule. In certain implementations, a molar ratio of the cationic lipids to the cholesterol to the phospholipid to the polyethylene glycol modified lipid molecule may be (45-55):(5-15):(35-45):(0.5-2). In certain implementations, a molar ratio of the cationic lipids to the cholesterol to the phospholipid to the polyethylene glycol modified lipid molecule may be 50:10:38.5:1.5.

For example, the composition may contain the mRNA and the lipid nanoparticles, wherein the mRNA may encode an amino acid sequence shown in SEQ ID NO: 15, the lipid nanoparticles may include the cationic lipids, the cholesterol, the phospholipid and the polyethylene glycol modified lipid molecule, and a molar ratio of the cationic lipids to the cholesterol to the phospholipid to the polyethylene glycol modified lipid molecule may be 50:10:38.5:1.5.

For example, the composition may contain the mRNA and the lipid nanoparticles, wherein the mRNA may encode the amino acid sequence shown in SEQ ID NO: 15, the lipid nanoparticles may contain the DLin-MC3-DMA, the cholesterol, the DSPC and the PEG2000-DMG, and a molar ratio of the DLin-MC3-DMA to the cholesterol to the DSPC to the PEG2000-DMG may be 50:10:38.5:1.5.

For example, the composition may contain the mRNA and the lipid nanoparticles, wherein the mRNA may contain a nucleotide sequence shown in SEQ ID NO: 23, the lipid nanoparticles may contain the cationic lipids, the cholesterol, the phospholipid and the polyethylene glycol modified lipid molecule, and a molar ratio of the cationic lipids to the cholesterol to the phospholipid to the polyethylene glycol modified lipid molecule may be 50:10:38.5:1.5.

For example, the composition may contain the mRNA and the lipid nanoparticles, wherein the mRNA may contain the amino acid sequence shown in SEQ ID NO: 23, the lipid nanoparticles may contain the DLin-MC3-DMA, the cholesterol, the DSPC and the PEG2000-DMG, and a molar ratio of the DLin-MC3-DMA to the cholesterol to the DSPC to the PEG2000-DMG may be 50:10:38.5:1.5.

### Preparation Method

The present application provides a method for preparing a delivery vector in a composition. For example, multilamellar vesicles (MLVs) may be prepared through existing techniques, such as depositing selected lipids on inner walls of suitable containers or vessels; and dissolving the lipids in a suitable solvent and then evaporating the solvent to retain a thin film on an inner side of a vessel, or conducting spray-drying. A water phase may be added to a rotating vessel, leading to the formation of the MHLVs. Then, unilamellar vesicles (ULVs) can be formed by homogenization, ultrasound treatment, or extrusion of multilamellar vesicles. In addition, the unilamellar vesicles can be formed through a detergent removal technology.

In the present application, the composition contains the delivery vector (such as lipid nanoparticles), wherein an mRNA may be associated with a surface of the lipid delivery vector and encapsulated within it. For example, in preparing the composition of the present application, the delivery vector of cationic lipids may be associated with the mRNA through electrostatic interaction.

In certain embodiments, the composition contains diagnostic radioactive nuclides, fluorescent substances, or other substances that are detectable in vivo and in vitro.

In the present application, an appropriate size of the lipid delivery vector (such as the lipid nanoparticles) is selected considering the size of target cells or tissue and the degree of application of liposome to be prepared. In certain embodiments, an mRNA may be delivered to a specific cell or tissue. For example, in order to target liver cells, the size of the lipid delivery vector (such as the lipid nanoparticles) may be determined, so that its size is smaller than the fenestrated gap of the endothelial lining hepatic sinusoid in the liver, allowing the lipid delivery vector (such as the lipid nanoparticles) to easily penetrate these endothelial fenestrated gaps to reach the target liver cells. The lipid delivery vector (such as the lipid nanoparticles) may have a sufficiently large diameter to limit or significantly avoid distribution within certain cells or tissue. In the present application, the size (e.g. diameter) of the lipid delivery vector (such as the lipid nanoparticles) may be in the range of about 25 to 250 nm, such as less than about 250 nm, 175 nm, 150 nm, 125 nm, 100 nm, 75 nm, 50 nm, 25 nm, or 10 nm. For example, the size (e.g. diameter) of the lipid delivery vector (such as the lipid nanoparticles) may be in the range of about 25 to 250 nm, such as about 50 to 200 nm, about 75 to 175 nm, about 75 to 150 nm, or about 75 to 125 nm.

### Vector, Cell and Pharmaceutical Composition

In another aspect, the present application provides a vector, the vector containing a nucleic acid molecule. For example, the vector may be a viral vector, such as an adenovirus vector, an adeno-associated virus vector, and/or a lentivirus vector.

In another aspect, the present application provides a cell, the cell containing the nucleic acid molecule and/or the vector. In the present application, the cell may be a prokaryocyte, such as E. coli. In the present application, the cell may be a eukaryocyte, such as a yeast cell, an insect cell, a plant cell and an animal cell. In the present application, the cell may be a mammal cell, such as a mouse cell and a human cell. In the present application, the cell may be a 293T cell or a P12 cell.

In another aspect, the present application provides a method for preparing a recombinant protein, and the method includes culturing the cell under a condition of expressing the recombinant protein.

In another aspect, the present application provides a pharmaceutical composition, and the pharmaceutical composition includes the recombinant protein, the nucleic acid molecule, the composition, the vector and/or the cell, and optionally a pharmaceutically acceptable carrier.

In another aspect, the present application provides a kit or a drug administration device, and the kit or the drug administration device includes the recombinant protein, the nucleic acid molecule, the composition, the vector, the cell, and/or the pharmaceutical composition. The recombinant protein, the nucleic acid molecule, the composition, the vector, the cell and/or the pharmaceutical composition of the present application may be included in a single common container, and they may also be optionally combined with one or more therapeutic agents and optionally prepared together in the kit.

### Therapeutic Method

In another aspect, the present application provides a method for relieving, preventing and/or treating central and/or peripheral nervous system diseases. The method includes administrating to a subject in need the recombinant protein, the nucleic acid molecule, the composition, the vector, the cell, and/or the pharmaceutical composition. For example, the central and/or peripheral nervous system diseases may include neurodegenerative diseases. For example, the central and/or peripheral nervous system diseases may include peripheral neuropathy. For example, the peripheral neuropathy includes toxic peripheral neuropathy, hereditary peripheral neuropathy and/or metabolic peripheral neuropathy. For example, the central and/or peripheral nervous system diseases may be included in certain implementations, and the peripheral neuropathy includes toxic peripheral neuropathy, hereditary peripheral neuropathy and/or metabolic peripheral neuropathy.

In certain implementations, the central and/or peripheral nervous system diseases include diabetic peripheral neuropathy, drug-related peripheral neuropathy, hereditary motor sensory neuropathy, post-traumatic repair of peripheral nerves, traumatic optic neuritis and/or Alzheimer's disease.

In another aspect, the present application provides a use of the recombinant protein, the nucleic acid molecule, the composition, the vector, the cell and/or the pharmaceutical composition in preparation of a medication which may be used for relieving, preventing and/or treating central and/or peripheral nervous system diseases. For example, the central and/or peripheral nervous system diseases may include neurodegenerative diseases. For example, the central and/or peripheral nervous system diseases may include peripheral neuropathy. For example, the peripheral neuropathy includes toxic peripheral neuropathy, hereditary peripheral neuropathy and/or metabolic peripheral neuropathy. For example, the central and/or peripheral nervous system diseases may be included in certain implementations, and the peripheral neuropathy includes toxic peripheral neuropathy, hereditary peripheral neuropathy and/or metabolic peripheral neuropathy.

In another aspect, the present application provides a method for promoting secretion and/or expression of a nerve growth factor (NGF) protein. The method includes using the recombinant protein, the nucleic acid molecule, the composition, the vector, the cell, and/or the pharmaceutical composition. For example, the method may be a non-therapeutic method. For example, the method may be an in-vitro and/or ex-vivo method.

In another aspect, the present application provides a method for promoting proliferation and/or differentiation of nerve cells. The method includes using the recombinant protein, the nucleic acid molecule, the composition, the vector, the cell, and/or the pharmaceutical composition. For example, the method may be a non-therapeutic method. For example, the method may be an in-vitro and/or ex-vivo method.

In another aspect, the present application provides a method for repairing injured nerve cells. The method includes using the recombinant protein, the nucleic acid molecule, the composition, the vector, the cell, and/or the pharmaceutical composition. For example, the method may be a non-therapeutic method. For example, the method may be an in-vitro and/or ex-vivo method.

Without being limited by any theory, the examples in the following are only intended to illustrate various technical solutions of the present application, and are not intended to limit the scope of the present application.

### Example

### Example 1 Design Optimization and Synthesis of NGF^{R100W} mRNA

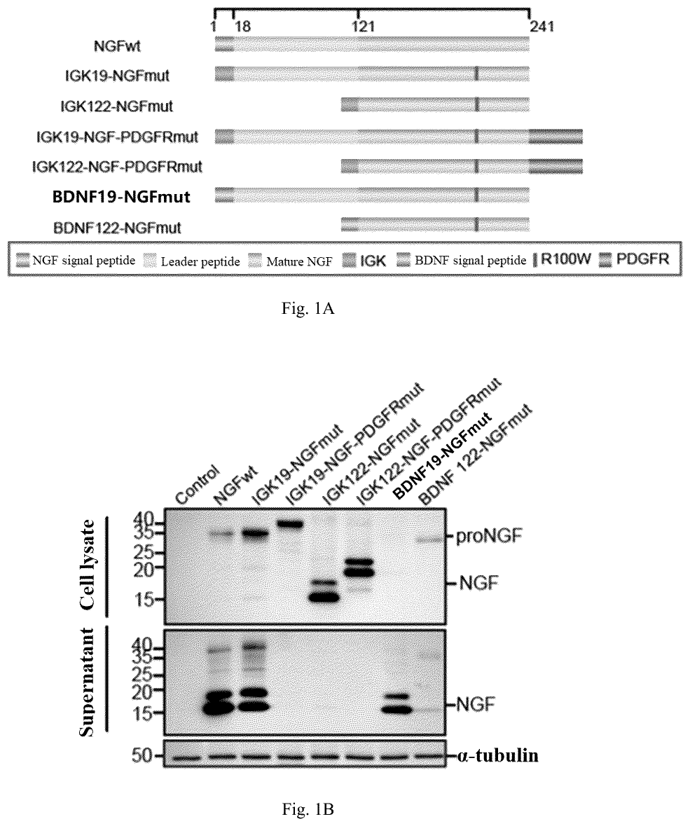

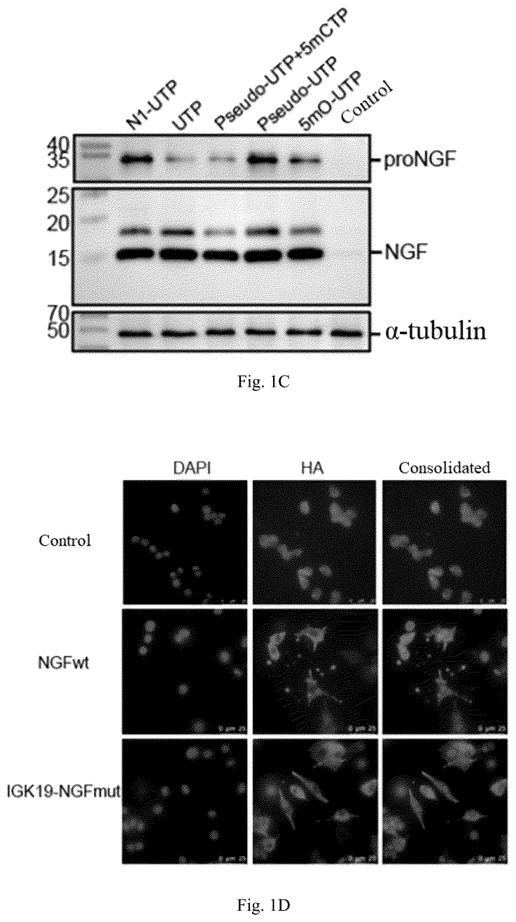

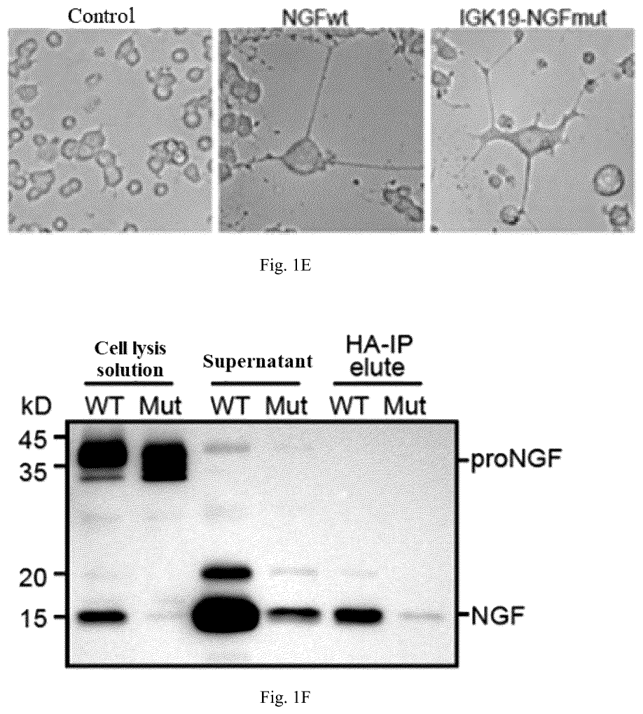

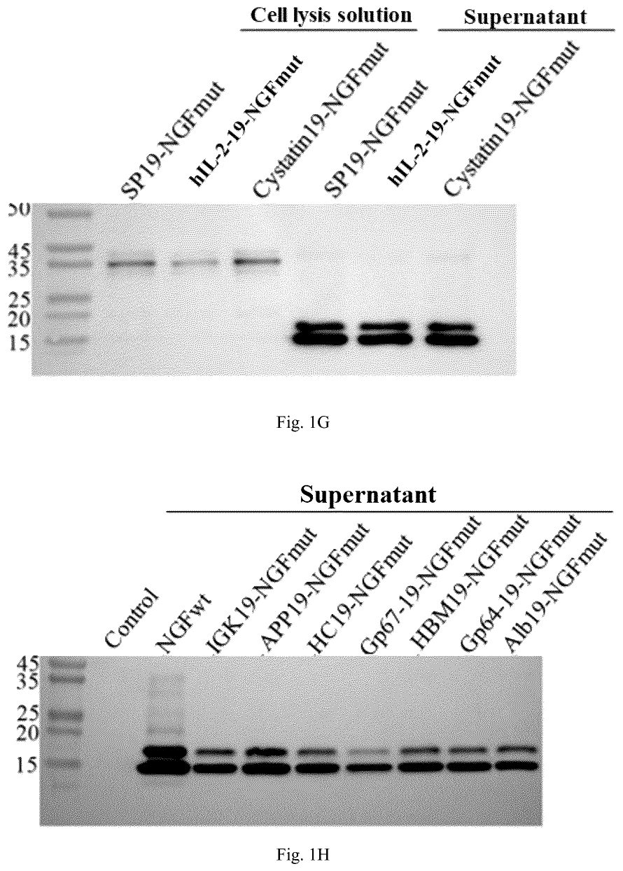

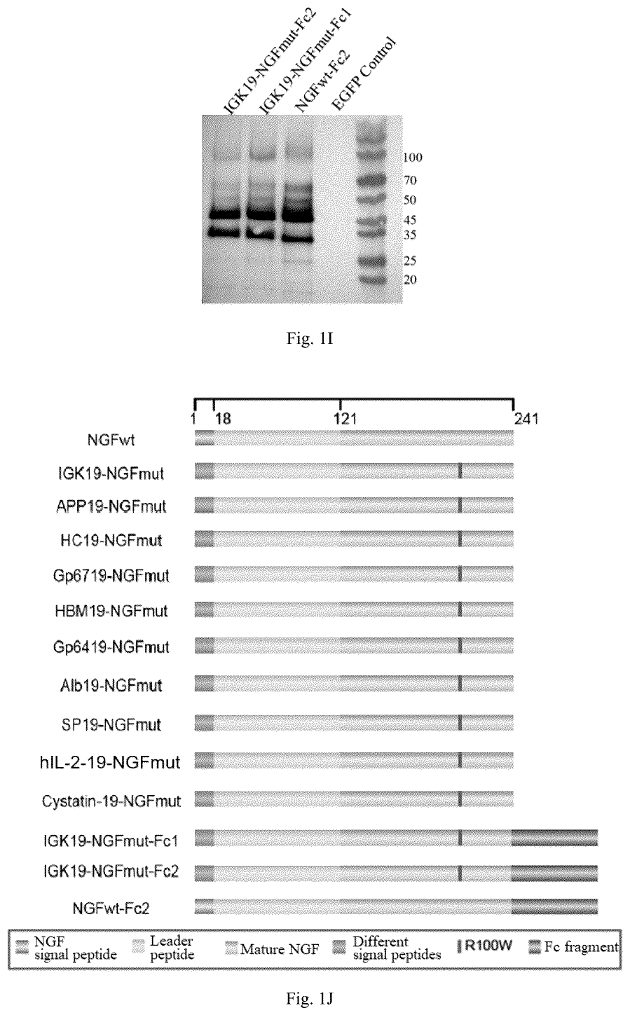

A recombinant protein containing an NGF mutant was constructed by replacing an NGF endogenous signal peptide with an exogenous signal peptide, truncating a leader peptide, and increasing transmembrane anchoring regions, etc. as shown in FIG. 1A or FIG. 1J. NGFwt: a full-length wild type NGF protein, from an N terminal to a C terminal, being NGF signal peptide+leader peptide+mature NGF protein sequentially; NGFmut: a full-length NGF^{R221W} mutant protein, from an N terminal to a C terminal, being NGF signal peptide+leader peptide+mature NGF^{R100W} mutant protein sequentially; IGK19-NGFmut: from an N terminal to a C terminal, being IGK signal peptide+leader peptide+mature NGF^{R100W} mutant protein sequentially; IGK122-NGFmut: from an N terminal to a C terminal, being IGK signal peptide+mature NGF^{R100W} mutant protein sequentially; IGK19-NGF-PDGFRmut: from an N terminal to a C terminal, being IGK signal peptide+leader peptide+mature NGF^{R100W} mutant protein+PDGFR transmembrane anchoring region sequentially; IGK122-NGF-PDGFRmut: from an N terminal to a C terminal, being IGK signal peptide+mature NGF^{R100W} mutant protein+PDGFR transmembrane anchoring region sequentially; BDNF19NGFmut: from an N terminal to a C terminal, being BDNF signal peptide+leader peptide+mature NGF^{R100W} mutant protein sequentially; BDNF122-NGFmut: from an N terminal to a C terminal, being BDNF signal peptide+mature NGF^{R100W} mutant protein sequentially; and amino acid sequences and nucleotide sequences were as shown in Table 1 below.

TABLE 1
Sequences of NGF or its mutant recombinant proteins

| | | Coding | Codon- | |
|---|---|---|---|---|
| | Amino | region | optimized | mRNA |
| | acid | sequence | coding region | sequence |
| | sequence | (SEQ | sequence | (SEQ |
| | (SEQ ID | ID | (SEQ | ID |
| Recombinant protein | NO.) | NO.) | ID NO.) | NO.) |
| NGFwt | 13 | 9 | 1 | 21 |
| NGFmut | 14 | 10 | 2 | 22 |
| IGK19-NGFmut | 15 | 11 | 3 | 23 |
| IGK122-NGFmut | 16 | — | 4 | 24 |
| IGK19-NGF-PDGFRmut | 17 | — | 5 | 25 |
| IGK122-NGF-PDGFRmut | 18 | — | 6 | 26 |
| BDNF19-NGFmut | 19 | 12 | 7 | 27 |
| BDNF122-NGFmut | 20 | — | 8 | 28 |
| IGK19-NGFmut-Fc1 | 54 | — | 46 | 50 |
| IGK19-NGFmut-Fc2 | 55 | — | 47 | 51 |
| SP-19-NGFmut | 58 | — | — | — |
| hIL-2-19-NGFmut | 59 | — | — | — |
| Cystatin 19-NGFmut | 60 | — | — | — |
| APP-19-NGFmut | 61 | — | — | — |
| HC-19-NGFmut | 62 | — | — | — |
| Gp67-19-NGFmut | 63 | — | — | — |
| HBM-19-NGFmut | 64 | — | — | — |
| Gp64-19-NGFmut | 65 | — | — | — |
| Alb-19-NGFmut | 66 | — | — | — |

The DNA sequence of the above coding region was obtained by in vitro transcription using T7 polymerase. HA tags were added at the 3′ terminal and cloned downstream of a T7 promoter on a pSG5L vector. A universal primer for a linear DNA template for in vitro transcription was obtained on the vector, and the DNA template for in vitro transcription was obtained by PCR. The T7 RNA polymerase was used for in vitro transcription, after DNA enzymes were used for digesting the template, Escherichia coli poly(A) polymerase EPAP (E. coli Poly(A) Polymerase) was used for poly(A) tail addition, and finally an mRNA was obtained through purification recovery.

The mRNA was expressed in a 293T tool cell line and Western blot was used to detect an expression amount. The results were shown in FIG. 1B, and protein expression was detected in supernatants of IGK19-NGFmut and BDNF19-NGFmut. FIG. 1F showed that, compared with the wild type NGF protein (WT), the content of secretion of an NGF^{R100W} protein (Mut) without a heterologous signal peptide to a supernatant was relatively low, mostly existing in a cell in the form of a proNGF. In addition, with several other common signal peptides (FIG. 1J), such as APP (azurocidin preproprotein), HC (immunoglobulin heavy chain), SP (signal peptide), Cystatin (Cystatin-S precursor) HBM, Gp64 and Alb (albumin), as shown in FIGS. 1G to 1I, the results showed that they all could promote excretion of the NGFmut to a certain extent.

### Example 2 Detection of Expression Amount of mRNA-Modified Proteins

In this example, an IGK19-NGFmut mRNA was modified, that is, nucleotides carrying different chemical modifications were added respectively during transcription to obtain mRNAs carrying chemical modifications, where the introduction of chemical modification nucleotides was 100% replacement. The chemical modifications used were N1-methylpseudouridine-5′-triphosphate (N1-Methylpseudo-UTP, namely N1-UTP), pseudouridine-5′-triphosphate (pseudo-UTP), 5-methoxyuridine-5′-triphosphate (5-Methoxy-UTP, namely 5mO-UTP) and 5-methylcytidine-5′-triphosphate (5-Methyl-CTP, 5mCTP) respectively. The results were shown in FIG. 1C, a protein expression amount obtained from the translation of the IGK19-NGFmut mRNA with different modifications was equivalent, and the protein expression amount with the N1-UTP modification was the highest. The mRNA for subsequent biochemical and functional studies was modified with the N1-UTP.

### Example 3 Detection of Function of Promoting Nerve Cell Differentiation of NGF^{R100W}

A PC12 cell line was derived from transplantable mouse pheochromocytoma, and the cell had a reversible neuronal phenotype response to an NGF. When PC12 cells were exposed to an environment containing NGF proteins, they could respond within a week, which mainly stopped cell division and extended neural processes. The PC12 cells were cultured in an RPMI1640 medium containing 5% fetal bovine serum and 5% horse serum. When the cells reached the logarithmic growth phase, NGFwt or IGK19-NGFmut mRNA was used for transfection. After 24 hours, the cells were stained with DAPI and HA, and the morphology of the cells was observed under a fluorescence microscope and an electron microscope. As shown in FIGS. 1D and 1E, the results showed that the mRNA of IGK19NGFmut, like NGFwt, could be translated and expressed as an NGF^{R100W} mutant protein, and promote the differentiation of the PC12 cells.

### Example 4 Obtaining Lipid Nanoparticles by Encapsulating NGF mRNA with Lipids

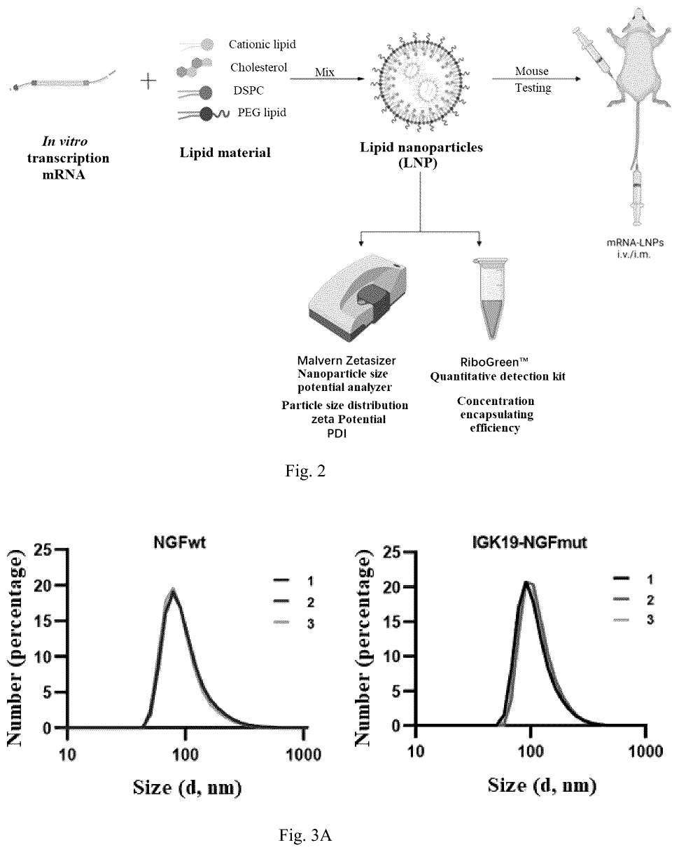

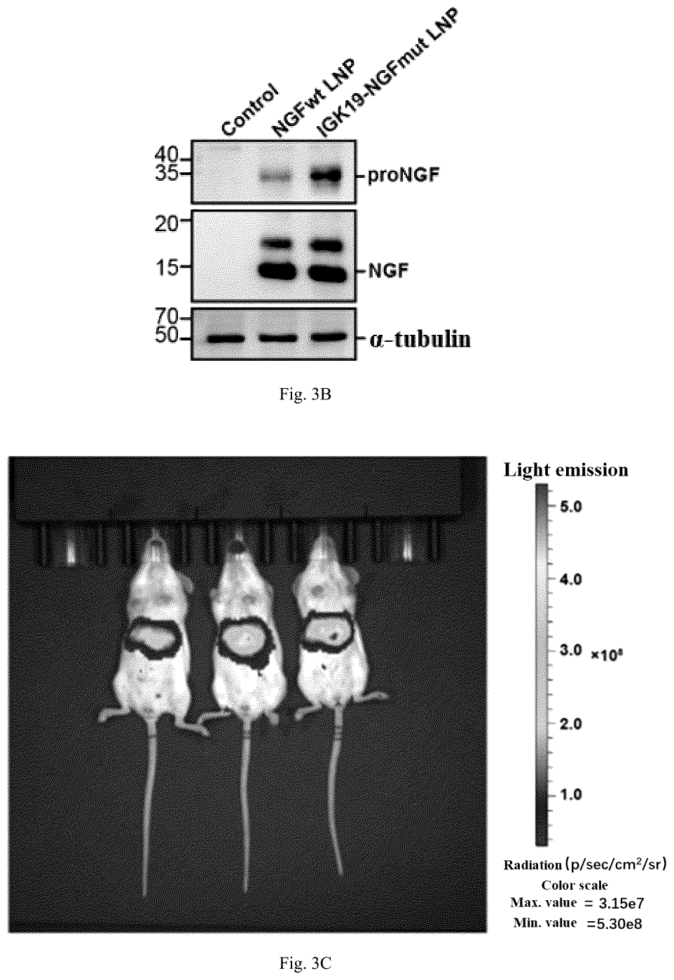

An NGF mRNA and lipid mixture were encapsulated into a lipid nanoparticle (LNP) system to deliver an mRNA, of which the process was as shown in FIG. 2. Four types of lipid molecules were used: cationic lipids (DLin-MC3-DMA), cholesterol, distearyl phosphatidylcholine (DSPC), and polyethylene glycol modified lipid molecules (PEG2000-DMG). The four types of lipids were dissolved in ethanol at a molar ratio of 50:10:38.5:1.5. The lipids dissolved in the ethanol were quickly mixed evenly with an mRNA transcribed in vitro through a T tube, and lipid nanoparticles with a diameter of about 100 nm were obtained by self-packaging. The NGFwt and IGK19-NGFmut transcribed in vitro were mixed and packaged with lipids in a ratio of mRNA: cationic lipids=1:3 (molar ratio) to form nanoparticles LNP. A particle size of the LNPs was detected using a Malvern Zetasizer nanoparticle size potential analyzer, and the results showed that the size of the NGFwt and IGK19-NGFmut were about 100 nm, as shown in FIG. 3A.

2 μg of NGF LNPs were directly dropwise added to 293 T cells for cell transfection, and the expression of mature NGF proteins excreted into a supernatant could be detected in the cells and the supernatant, as shown in FIG. 3B. A fluorescence reporting system in which luciferase oxidized D-luciferin was utilized to emit fluorescence, combined with the biological in vivo fluorescence imaging technology, to determine the localization and expression kinetics of expression of nanoparticles in mice. The mice were injected with nanoparticles encapsulating luciferase mRNAs through the tail vein, and then intraperitoneally injected with luciferin substrates at different time points, and the intensity and localization of biological light generated by oxidation of the substrates by the luciferase were detected by using small animal in vivo imaging. The results showed that the expression efficiency of the nanoparticles was higher when administered through a circulatory pathway, and it was mainly concentrated in the liver, as shown in FIG. 3C.

### Example 5 Validation of Low Pain-Inducing Effect of IGK19-NGFmut LNPs in Mice

Eight 8-week-old C57BL/6 male mice were divided into two groups, with four mice in each group, and by using plantar injection, each mouse was injected with 2 μg of IGK19-NGFmut LNPs and 2 μg of NGFwt LNPs in plantar foot pad areas of the hind paws of the mice. Mouse behavioral experiments were conducted 8 hours after injection to detect a pain threshold of heat pain in the soles of the mice. The mice were placed on a transparent rack 8 hours after plantar injection, the two groups of mice were given the same intensity of infrared heat source stimulation, and the latent time of paw lifting of the mice was recorded.

Ten 8-week-old C57BL/6 male mice were divided into two groups, with 5 mice in each group, and were injected with 2 μg of IGK19-NGFmut LNPs and 2 μg of NGFwt LNPs in the same way. The mice were placed on a grid frame 8 hours after plantar injection, mechanical stress of different magnitudes was applied to the soles of the mice using von frey filaments, and stress values that could cause the mice to lift their claws were recorded.

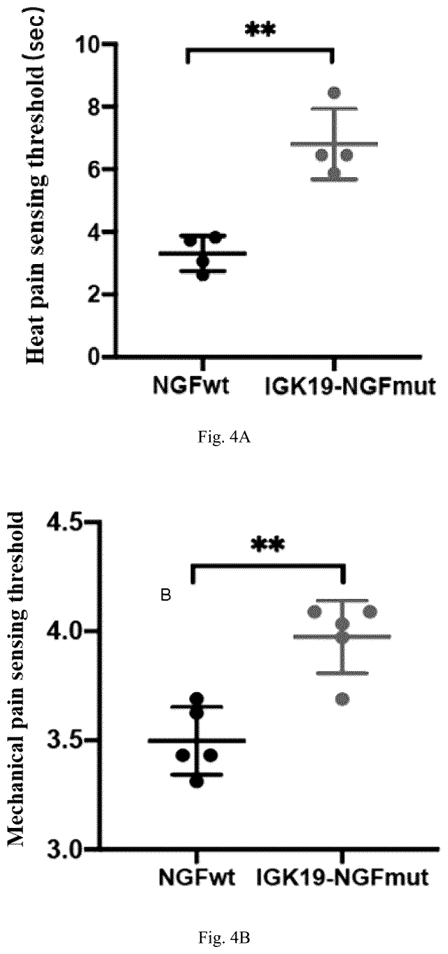

The results showed that the mice injected with the IGK19-NGFmut LNPs had a significantly higher sensitivity threshold to heat pain compared to the control group mice injected with the NGFwt LNPs, as shown in FIG. 4A; and meanwhile, FIG. 4B also showed that the expression of the IGK19-NGFmut mRNAs made the mice less sensitive in the response of mechanical pain compared to the expression of the NGFwt, indicating that the IGK19-NGFmut LNPs had a lower pain-inducing effect on mice compared to the expression of the NGFwt LNPs in the soles of the mice.

### Example 6 Validation of Nerve Repair Effect of IGK19-NGFmut LNPs in Mice with Peripheral Nerve Injuries

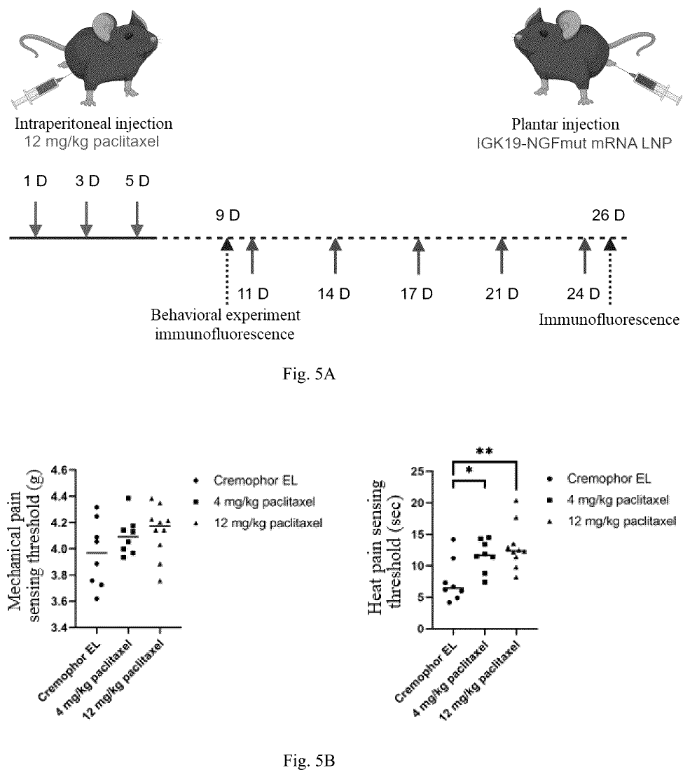

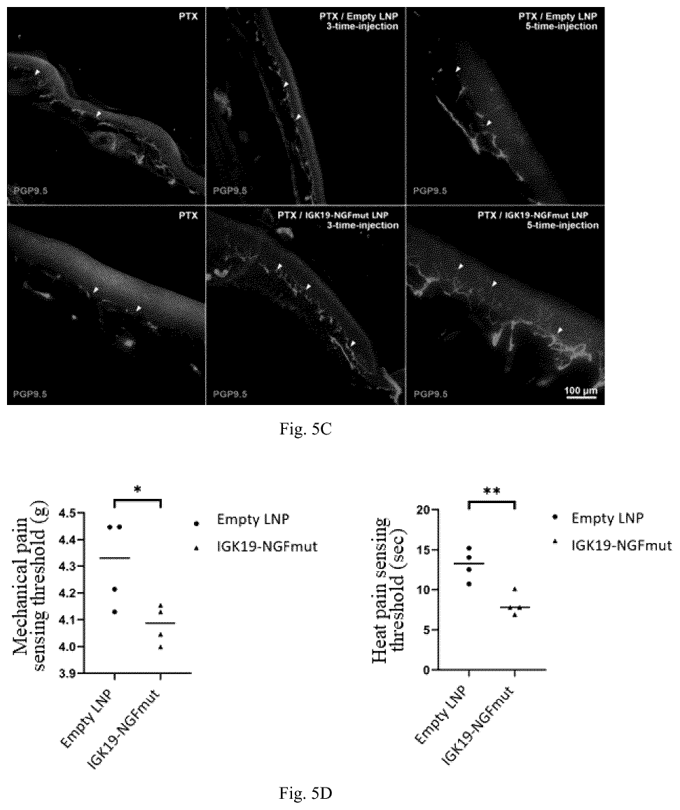

Modeling of mouse peripheral nerve injuries was conducted with fifteen 8-week-old C57BL/6 male mice by using a method of intraperitoneal injection of paclitaxel every other day. One week after the completion of modeling, the mice were divided into three groups, with 6 mice in each group injected with IGK19-NGFmut LNPs and NGFwt LNPs, and only 3 mice in a modeling group. By using plantar injection, each mouse was injected with 2 kg of the IGK19-NGFmut LNPs and 2 kg of the NGFwt LNPs in the plantar foot pad area of the hind paws of the mice, and the injection was conducted every three days. After 5 injections, the pain threshold of heat pain and the pain threshold of mechanical pain in the soles of the mice were tested. The mice were placed on a grid frame, mechanical stress of different magnitudes was applied to the soles of the mice using von frey filaments, and stress values that could cause the mice to lift their claws were recorded. After completing the mechanical pain threshold test, a 2-hour buffer period was given, then the two groups of mice were placed on a hot plate at 55° C., and the latent time of paw lifting of the mice was recorded. The experimental process was as shown in FIG. 5A.

The results of heat pain and mechanical pain in FIG. 5B showed that the mouse peripheral nerve injuries caused by the injection dose of 12 mg/kg of paclitaxel could all cause an increase in plantar pain threshold of the mice. As the number of injections increased, the continuity and density of peripheral nerves of the mice injected with the IGK19-NGFmut LNPs could be increased obviously compared to the control group mice injected with the NGFwt LNPs, as shown in FIG. 5C; and meanwhile, FIG. 5D also showed that the expression of the IGK19-NGFmut mRNAs made the mice more sensitive in the response of mechanical pain and heat pain compared to the expression of NGFwt, indicating that the expression of the IGK19-NGFmut LNPs in the soles of the mice could repair peripheral nerve injuries caused by paclitaxel.

The above detailed explanation is provided in the form of explanation and examples, and is not intended to limit the scope of the attached claims. At present, the various changes in the implementations listed in the present application are obvious to those of ordinary skill in the art, and are retained within the scope of the attached claims and their equivalent methods.

What is claimed is:

1. A recombinant protein, comprising a nerve growth factor (NGF) protein variant and a heterologous signal peptide linked to the NGF protein variant, wherein the amino acid of the recombinant protein is shown in SEQ ID NO: 15.

2-23. (canceled)

24. An isolated nucleic acid molecule encoding the recombinant protein according to claim 1; wherein the nucleic acid molecule is RNA;
wherein, the RNA comprises modifications at one or more positions selecting from: a 5′ cap, a 5′ untranslated region, an open reading frame, a 3′ untranslated region, and a poly A tail;
wherein the RNA comprises at least one modified nucleotide;
and the nucleotide sequence of the RNA is shown in SEQ ID NO: 23.

25-27. (canceled)

28. The nucleic acid molecule according to claim 24, wherein the modified nucleotide contained in the nucleic acid molecule comprises one or more nucleotides selecting from: N1-methylpseudouridine-5′-triphosphate (N1-Methylpseudo-UTP), pseudouridine-5′-triphosphate (pseudo-UTP) and 5-methoxyuridine-5′-triphosphate (5-Methoxy-UTP).

29-34. (canceled)

35. A composition comprising (a) an mRNA consisting of a nucleotide sequence shown in SEQ ID NO: 23, and (b) a delivery vector;
wherein, the mRNA comprises modifications at one or more positions selecting from: a 5′ cap, a 5′ untranslated region, an open reading frame, a 3′ untranslated region, and a poly A tail;
and the mRNA comprises at least one modified nucleotide.

36-62. (canceled)

63. The composition according to claim 35, wherein the modified nucleotide contained in the mRNA comprises one or more nucleotides selecting from: N1-methylpseudouridine-5′-triphosphate (N1-Methylpseudo-UTP), pseudouridine-5′-triphosphate (pseudo-UTP) and 5-methoxyuridine-5′-triphosphate (5-Methoxy-UTP).

64-81. (canceled)

82. The composition according to claim 35, wherein the delivery vector comprises the DLin-MC3-DMA, the cholesterol, the DSPC, and PEG2000-DMG, and a molar ratio of the DLin-MC3-DMA to the cholesterol to the DSPC to the PEG2000-DMG is 50:38.5:10:1.5.

83. (canceled)

84. The composition according to claim 35, wherein the delivery vector has a diameter of about 80 nm to about 200 nm.

85. The composition according to claim 35, wherein the mRNA is encapsulated in the delivery vector.

86. A vector, comprising the nucleic acid molecule according to claim 24.

87. A cell, expressing the recombinant protein according to claim 1.

88. A method for preparing a recombinant protein, comprising culturing the cell expressing the recombinant protein according to claim 1.

89. A pharmaceutical composition, comprising the recombinant protein according to claim 1, and optionally a pharmaceutically acceptable carrier.

90. A kit or a drug administration device, comprising the recombinant protein according to claim 1.

91-100. (canceled)

101. A method for promoting secretion or expression of a nerve growth factor (NGF) protein, comprising using the recombinant protein according to claim 1;
wherein the method is a non-therapeutic method;
and the method is an in-vitro or ex-vivo method.

102-103. (canceled)

104. A method for promoting proliferation or differentiation of nerve cells, comprising using the recombinant protein according to claim 1;
wherein the method is a non-therapeutic method;
and the method is an in-vitro or ex-vivo method.

105-109. (canceled)
